(19) United States
(12) Patent Application Publication
(10) Pub. No.: US 2024/0124608 A1
(43) Pub. Date: Apr. 18, 2024

(54) METHODS AND MATERIALS FOR TREATING CLONAL T CELL EXPANSIONS

(71) Applicant: The Johns Hopkins University, Baltimore, MD (US)

(72) Inventors: Michael S. Hwang, Seattle, WA (US); Kenneth W. Kinzler, Frankford, DE (US); Brian J. Mog, Baltimore, MD (US); Nickolas Papadopoulos, Towson, MD (US); Andrew M. Pardoll, Baltimore, MD (US); Suman Paul, Baltimore, MD (US); Bert Vogelstein, Baltimore, MD (US); Shibin Zhou, Owings Mills, MD (US)

(21) Appl. No.: 18/277,631

(22) PCT Filed: Feb. 15, 2022
(86) PCT No.: PCT/US2022/016423

§ 371 Date: Aug. 17, 2023

Related U.S. Application Data
(60) Provisional application No. 63/150,232, filed on Feb. 17, 2021.

(51) Int. Cl. C07K 16/30; A61P 35/00; C07K 16/28; A61K 39/00

(52) U.S. Cl. CPC C07K 16/3061; A61P 35/00; C07K 16/2809; A61K 2039/505; C07K 2317/31; C07K 2317/54; C07K 2317/55; C07K 2317/622; C07K 2317/626; C07K 2317/73

(57) ABSTRACT

This document relates to methods and materials for treating T cell cancers. For example, a composition containing one or more bispecific molecules targeting T cell receptor £ chain constant region (TRBC) can be administered to a mammal having a T cell cancer to treat the mammal. For example, this document provides methods and materials for using one or more bispecific molecules to treat a mammal having a T cell cancer.

### CROSS-REFERENCE To RELATED APPLICATIONS

This application claims the benefit of U.S. Patent Application Ser. No. 63/150,232, filed on Feb. 17, 2021. The disclosure of the prior application is considered part of (and is incorporated by reference in) the disclosure of this application.

### STATEMENT REGARDING FEDERAL FUNDING

This invention was made with government support under grants AR048522, CA006973, CA009071, CA06292, CA230400 and GM007309 awarded by the National Institutes of Health. The government has certain rights in the invention.

### SEQUENCE LISTING

This document contains a Sequence Listing that has been submitted electronically as an ASCII text file named 44807-0386WO1_ST25.txt. The ASCII text file, created on Feb. 10, 2022, is 24 kilobytes in size. The material in the ASCII text file is hereby incorporated by reference in its entirety.

### BACKGROUND

### 1. Technical Field

This document relates to methods and materials for treating clonal T cell expansions (e.g., pathogenic clonal T cell expansions such as T cell cancers). For example, a composition containing one or more bispecific molecules can be administered to a mammal having a T cell cancer to treat the mammal. For example, this document provides methods and materials for using one or more bispecific molecules to treat a mammal having a T cell cancer.

### 2. Background Information

T cell cancers are a heterogeneous group of malignancies that comprises about 15% of non-Hodgkin's lymphomas (Swerdlow et al., Blood 127:2375-2390 (2016)) and 20% of acute lymphoblastic leukemias (ALL; Han et al., Cancer Causes & Control 19:841-858 (2008); and Dores et al., Blood 119:34-43 (2012). Outcomes of T cell lymphomas and relapsed T cell ALL (T-ALL) are worse than those for equivalent B cell malignancies, with an estimated 5-year survival of only 32% in T cell lymphomas (Weisenburger et al., Blood 117:3402-3408 (2011)) and 7% in relapsed T-ALL (Fielding et al., Blood 109:944-950 (2007)).

Malignant B or T cells do not express cell-surface antigens that are distinct from their non-cancerous counterparts. There are several targeted immunotherapeutic agents for B cell malignancies that target pan-B cell antigens such as CD19 or CD20, which is feasible because the associated normal B cell aplasia is clinically well tolerated. However, a similar strategy targeting pan-T cell antigens is not feasible because the resultant T cell depletion would lead to a clinically unacceptable level of immunosuppression.

### SUMMARY

VDJ recombination, combined with allelic exclusion, results in expression of only one of the 2 TRBC polypeptides (i.e., TRBC1 polypeptides or TRBC2 polypeptides) on the surface of each T cell. Normal healthy T cells express a mixture of both TRBC1 and TRBC2. In contrast, clonal T cell cancers express only one of the two TCR β chain constant regions (e.g., express only TRBC1 or TRBC2). As described herein, bispecific molecules targeting a TRBC polypeptide can selectively deplete only one of the 2 TRBC polypeptides while sparing the other of the 2 TRBC polypeptides. For example a bispecific molecule targeting a TRBC1 polypeptide can selectively deplete TRBC1⁺ T cells (e.g., cancerous TRBC1+ T cells and healthy TRBC1⁺ T cells) while sparing the TRBC2⁺ healthy human T cells (see, e.g., FIG. 1A) such that the remaining healthy TRBC2⁺ T cells are sufficient to maintain a functioning immune system.

This document provides methods and materials for treating clonal T cell expansions (e.g., pathogenic clonal T cell expansions such as T cell cancers). In some cases, this document provides bispecific molecules that can be used to treat T cell cancers. For example, a bispecific molecule that includes at least two antigen binding domains, where a first antigen binding domain (e.g., a first single-chain variable fragment (scFv)) can bind a T cell receptor (TCR) β chain constant region (TRBC) polypeptide and a second antigen binding domain (e.g., a second scFv) can bind the same TRBC polypeptide or can bind a T cell co-receptor polypeptide (e.g., a CD3 polypeptide), can be used to treat a mammal (e.g., a human) having a T cell cancer. In some cases, this document provides methods for treating T cell cancers. For example, one or more bispecific molecules provided herein (e.g., a composition containing one or more bispecific molecules provided herein) can be administered to a mammal having a T cell cancer to treat the mammal.

As described herein, clonal T cell expansions (e.g., pathogenic clonal T cell expansions such as T cell cancers) can be treated by targeting specific subsets of TCR antigens. For example, T cell cancers having a malignant expansion of TRBC1⁺ T cells can be treated using bispecific antibodies (BsAbs) targeting a TRBC1 polypeptide (e.g., BsAbs including first antigen binding domain that can bind a TRBC1 polypeptide and a second antigen binding domain that can bind the same TRBC1 polypeptide). BsAbs targeting TRBC1 can stimulate healthy T cells to specifically lyse TRBC1⁺ T cells (including malignant TRBC1⁺ cells) while preserving TRBC2⁺ T cells (e.g., approximately half of the normal T cells) within a mammal (see, e.g., FIG. 1A). Also as demonstrated herein, treatment with BsAbs targeting a TRBC1 polypeptide can be followed by treatment to target any residual malignant TRBC1⁺ T cells that may still remain. For example, BsAbs that can bind a TRBC1 polypeptide and can bind a CD3 polypeptide can be used to recruit healthy TRBC2⁺ T cells as effector T cells to target (e.g., target and destroy) the remaining malignant TRBC1⁺ T cells (see, e.g., FIG. 1B). For example, one or more TRBC1 targeting antibody drug conjugates (TRBC1-ADCs) can be used to target (e.g., target and destroy) the remaining malignant TRBC1⁺ T cells (see, e.g., FIG. 1C).

Similarly, T cell cancers having a malignant expansion of TRBC2⁺ T cells can be treated using BsAbs targeting a TRBC2 polypeptide (e.g., BsAbs including first antigen binding domain that can bind a TRBC2 polypeptide and a second antigen binding domain that can bind the same TRBC2 polypeptide), and such treatment with BsAbs targeting a TRBC2 polypeptide can optionally be followed by treatment with BsAbs that can bind a TRBC2 polypeptide and can bind a CD3 polypeptide and/or followed by treatment with one or more TRBC2-ADCs.

Having the ability to treat clonal T cell expansions (e.g., T cell cancers) as described herein (e.g., by administering one or more bispecific molecules including a first antigen binding domain that can bind a TRBC polypeptide and a second antigen binding domain that can bind the same TRBC polypeptide, and, optionally, one or more bispecific molecules including a first antigen binding domain that can bind a TRBC polypeptide and a second antigen binding domain that can bind a T cell co-receptor polypeptide such as a CD3 polypeptide) provides a unique and unrealized opportunity to selectively deplete clonal T cell cancers while retaining approximately half of the normal T cells (e.g., enough healthy T cells to maintain adequate T cell immunity and a functioning immune system). Additionally, bispecific molecules provided herein (e.g., bispecific molecules including a first antigen binding domain that can bind a TRBC polypeptide and a second antigen binding domain that can bind the same TRBC polypeptide and, optionally, bispecific molecules including a first antigen binding domain that can bind a TRBC polypeptide and a second antigen binding domain that can bind a T cell co-receptor polypeptide such as a CD3 polypeptide) can be used as a cost-effective, off-the-shelf targeted therapeutic for T cell cancers.

In general, one aspect of this document features bispecific molecules including a polypeptide comprising a first antigen binding domain that can bind a TRBC polypeptide and a polypeptide comprising a second antigen binding domain that can bind the TRBC polypeptide. The polypeptide comprising the first antigen binding domain that can bind the TRBC polypeptide and the polypeptide comprising the second antigen binding domain that can bind the TRBC polypeptide can each be independently a single-chain variable fragment (scFv), an antigen-binding fragment (Fab), a F(ab′)2 fragment, or any biologically active fragment thereof. The binding affinity of the first antigen binding domain that can bind the TRBC polypeptide can be lower than a binding affinity of the second antigen binding domain that can bind the TRBC polypeptide. The TRBC polypeptide can be a TRBC1 polypeptide or a TRBC2 polypeptide. The TRBC polypeptide can be a TRBC1 polypeptide. The first antigen binding domain that can bind to the TRBC1 polypeptide or the second antigen binding domain that can bind to the TRBC1 polypeptide can include a light chain including a V_{L} CDR1 having an amino acid sequence set forth in SEQ ID NO:1, a V_{L} CDR2 having an amino acid sequence set forth in SEQ ID NO:2, and a V_{L} CDR3 having an amino acid sequence set forth in SEQ ID NO:3; and can include a heavy chain including a V_{H} CDR1 having an amino acid sequence set forth in SEQ ID NO:4, a V_{H} CDR2 having an amino acid sequence set forth in SEQ ID NO:5, and a V_{H} CDR3 having an amino acid sequence set forth in SEQ ID NO:6. The light chain can include or consist essentially of an amino acid sequence set forth in SEQ ID NO:7, and the heavy chain can include or consist essentially of an amino acid sequence set forth in SEQ ID NO:8. The light chain can include or consist essentially of an amino acid sequence set forth in SEQ ID NO:48, and the heavy chain an include or consist essentially of an amino acid sequence set forth in SEQ ID NO:49. The bispecific molecule also can include a molecule that can improve the stability of the bispecific molecule.

In another aspect, this document features methods for treating a mammal having a T cell cancer. The methods can include, or consist essentially of, administering to a mammal having a T cell cancer mammal a bispecific molecule comprising: a polypeptide comprising a first antigen binding domain that can bind a TRBC polypeptide; and a polypeptide comprising a second antigen binding domain that can bind the TRBC polypeptide. The mammal can be a human. The T cell cancer can be a clonal T cell cancer. The T cell cancer can be an acute lymphoblastic leukemia (ALL), a peripheral T cell lymphomas (PTCL), an angioimmunoblastic T cell lymphomas (AITL), a T cell prolymphocytic leukemia (T-PLL), an adult T cell leukemia/lymphoma (ATLL), an enteropathy-associated T-cell lymphoma (EATL), a monomorphic epitheliotropic intestinal T-cell lymphoma (MEITL), a follicular T-cell lymphoma (FTCL), a nodal peripheral T-cell lymphoma (nodal PTCL), a cutaneous T cell lymphomas (CTCL), an anaplastic large cell lymphoma (ALCL), a T-cell large granular lymphocytic leukemia (T-LGL), an extra nodal NK/T-Cell lymphoma (NKTL), or a hepatosplenic T-cell lymphoma. The cancer cells within the mammal can be reduced by at least 50 percent. The method can be effective to improve survival of the mammal. The method also can include administering to the mammal, after the administration of the bispecific molecule, a second bispecific molecule comprising: a polypeptide comprising a third antigen binding domain that can bind the TRBC polypeptide; and a polypeptide comprising an antigen binding domain that can bind a CD3 polypeptide. The CD3 polypeptide can be a CD3γ polypeptide, a CD3δ polypeptide, or a CD3ε polypeptide. The method also can include administering to the mammal, after the administration of the bispecific molecule, a molecule comprising: a polypeptide comprising a third antigen binding domain that can bind the TRBC polypeptide; and an anti-cancer agent.

In another aspect, this document features methods for treating a mammal having a T cell cancer. The methods can include, or consist essentially of, administering to a mammal having a T cell cancer a first bispecific molecule comprising: a polypeptide comprising a first antigen binding domain that can bind a TRBC polypeptide and a polypeptide comprising a second antigen binding domain that can bind the TRBC polypeptide; and administering to the mammal a second bispecific molecule comprising: a polypeptide comprising a third antigen binding domain that can bind the TRBC polypeptide and a polypeptide comprising an antigen binding domain that can bind a CD3 polypeptide. The CD3 polypeptide can be a CD3γ polypeptide, a CD3δ polypeptide, or a CD3ε polypeptide. The mammal can be a human. The T cell cancer can be a clonal T cell cancer. The T cell cancer can be an ALL, a PTCL, an AITL, a T-PLL, an ATLL, an EATL, a MEITL, a FTCL, a nodal PTCL, a CTCL, an ALCL, a T-LGL, an NKTL, or a hepatosplenic T-cell lymphoma. The cancer cells within the mammal can be reduced by at least 50 percent. The method can be effective to improve survival of the mammal.

In another aspect, this document features methods for method for treating a mammal having a T cell cancer. The methods can include, or consist essentially of, administering to a mammal having a T cell cancer a first bispecific molecule comprising: a polypeptide comprising a first antigen binding domain that can bind a TRBC polypeptide and a polypeptide comprising a second antigen binding domain that can bind said TRBC polypeptide; and administering to the mammal a molecule comprising: a polypeptide comprising a third antigen binding domain that can bind the TRBC polypeptide and an anti-cancer agent. The mammal can be a human. The T cell cancer can be a clonal T cell cancer. The T cell cancer can be an ALL, a PTCL, an AITL, a PLL, an ATLL, an EATL, a MEITL, a FTCL, a nodal PTCL, a CTCL, an ALCL, a T-LGL, an NKTL, or a hepatosplenic T-cell lymphoma. The cancer cells within the mammal can be reduced by at least 50 percent. The method can be effective to improve survival of the mammal.

In another aspect, this document features methods for method for treating a mammal having a disease, disorder, or condition associated with a clonal T cell expansion. The methods can include, or consist essentially of, administering to a mammal having a disease, disorder, or condition associated with a clonal T cell expansion a bispecific molecule comprising: a polypeptide comprising a first antigen binding domain that can bind a TRBC polypeptide and a polypeptide comprising a second antigen binding domain that can bind the TRBC polypeptide. The mammal can be a human. The disease, disorder, or condition associated with a clonal T cell expansion can be graft versus host disease (GVHD), celiac disease, Felty's syndrome, Sjogren's syndrome, scleroderma, eosinophilic fasciitis, scleromyxedema, myositis, multiple sclerosis, Rasmussen's encephalitis, autoimmune thyroid diseases, neuromyelitis optica, aplastic anemia, paroxysmal nocturnal hemoglobinuria, Alzheimer's disease, narcolepsy, or aging. The method also can include administering to the mammal, after the administration of the bispecific molecule, a second bispecific molecule comprising: a polypeptide comprising a third antigen binding domain that can bind the TRBC polypeptide and a polypeptide comprising an antigen binding domain that can bind a CD3 polypeptide. The CD3 polypeptide can be a CD3γ polypeptide, a CD3δ polypeptide, or a CD3ε polypeptide.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIGS. 1A-1C: Illustration depicting the proposed selective TRBC1 depletion strategy. FIG. 1A) Healthy human T cells comprises 2 TRBC families, TRBC1⁺ and TRBC2⁺. Similarly, malignant clonal T cells are TRBC1⁺. TRBC1-TRBC1 bispecific antibody links healthy TRBC1⁺ cells with malignant TRBC1⁺ cells as well as healthy TRBC1⁺ T cells with other healthy TRBC1⁺ T cells, leading to selective killing of the malignant and healthy TRBC1⁺ populations while sparing the healthy TRBC2⁺ T cells. FIG. 1B) Post-TRBC1-TRBC1 bispecific antibody treatment, residual malignant TRBC1⁺ T cells may persists while the healthy TRBC1⁺ effector cells are depleted. A “mop up” strategy involves a subsequent TRBC1-CD3 bispecific antibody treatment that redirects the healthy TRBC2⁺ effector T cells to kill the residual malignant TRBC1⁺ T cells. FIG. 1C) Post-TRBC1-TRBC1 bispecific antibody treatment, residual malignant TRBC1⁺ cells may persists while the healthy TRBC1⁺ effector cells are depleted. The second “mop up” strategy involves a subsequent TRBC1-antibody drug conjugate (ADC) molecule treatment where the TRBC1 targeting ADC can bind to and kill the residual malignant TRBC1⁺ T cells, while the healthy TRBC2⁺ cells persist.

FIGS. 2A-2C: anti-TRBC antibody structures. FIGS. 2A-2B). Bispecific antibody structures. FIG. 2A) An exemplary TRBC1-CD3 bispecific antibody composed of αTRBC1 scFv linked with αCD3 scFv. FIG. 2B) An exemplary TRBC1-TRBC1 bispecific antibody composed of a αTRBC1 scFv linked to a second αTRBC1 scFv. FIG. 2C) An exemplary αTRBC1-ADC.

FIG. 3: Illustration depicting the arrangement of the variable light (VL) chain shown in green, variable heavy (VH) chain shown in orange, short peptide linker (G₄S), long peptide linker ((G₄S)₃) and the poly-histidine tail (H) shown in yellow, in the four TRBC1-TRBC1 bispecific antibody constructs. scDb: single chain diabody. BITE: bispecific T cell engager.

FIGS. 4A-4B: Expression of the four TRBC1-TRBC1 bispecific antibody constructs. The four TRBC1-TRBC1 bispecific antibodies were purified and analyzed using stain-free polyacrylamide gel electrophoresis (FIG. 4A) or western blot (FIG. 4B) with rabbit anti-6×His and HRP-conjugated anti-rabbit antibodies.

FIGS. 5A-5B: Binding of TRBC1-TRBC1 bispecific antibodies. FIG. 5A) Histograms showing binding of the four TRBC1-TRBC1 bispecific antibodies (#1, #2, #3 and #4) to Jurkat cells (express TRBC1), HPB-ALL cells (express TRBC2), Jurkat cells with T cell receptor knock out (TCR-KO), and healthy human T cells (ABO4 donor, express both TRBC1 and TRBC2). FIG. 5B) Histogram showing binding of the four TRBC1-TRBC1 bispecific antibodies along with the TRBC1-CD3 bispecific antibody (αC1-CD3) to Jurkat cells, HPB-ALL cells, Jurkat TCR-KO cells and AB04 healthy human T cells.

FIG. 6: TRBC1-TRBC1 and TRBC-CD3 bispecific antibodies induce T cell interferon gamma (IFNγ) release against T cell cancer cell lines in vitro. 5×10⁴ normal human T cells (from human donor AB04) were incubated with 5×10⁴ of the indicated target T cell cancer cell lines (Jurkat or HPB-ALL cells) in the presence of TRBC1-TRBC1 #3 (anti-C1 #3), or TRBC1-TRBC1 #4 (anti-C1 #4) or TRBC1-CD3 (anti-C1-CD3) at 5 ng/mL or 50 ng/mL for 17 hours. T cell cytokine release was then assessed by IFNγ ELISA.

FIG. 7: 5×10⁴ normal human T cells were incubated with TRBC1-TRBC1 #3, or TRBC1-TRBC1 #4 or TRBC-CD3 bispecific antibody (50 ng/ml) for 17 hours. 10 μL precision counting beads were added and flow cytometry was used to assess number of TRBC1-PE and GFP expressing cells. 500 beads were collected in each condition. Numbers beside dot plot indicate number of surviving cells.

FIG. 8: 5×10⁴ normal human T cells were incubated with 5×10⁴ wild-type Jurkat-GFP cells in presence of TRBC1-TRBC1 #3, or TRBC1-TRBC1 #4 or TRBC-CD3 bispecific antibody (50 ng/ml) for 17 hours. 10 μL precision counting beads were added and flow cytometry was used to assess number of TRBC1-PE and GFP expressing cells. 500 beads were collected in each condition. Numbers beside dot plot indicate number of surviving cells.

FIG. 9: 5×10⁴ normal human T cells were incubated with 5×10⁴ wild-type HPB-ALL-GFP cells in presence of TRBC1-TRBC1 #3, TRBC1-TRBC1 #4, or TRBC-CD3 bispecific antibody (50 ng/ml) for 17 hours. 10 μL precision counting beads were added and flow cytometry was used to assess number of TRBC1-PE and GFP expressing cells. 500 beads were collected in each condition. Numbers beside dot plot indicate number of surviving cells.

### DETAILED DESCRIPTION

This document provides methods and materials for treating clonal T cell expansions (e.g., pathogenic clonal T cell expansions such as T cell cancers). In some cases, this document provides bispecific molecules that can be used to treat T cell cancers. For example, this document provides bispecific molecules that include at least two antigen binding domains where a first antigen binding domain (e.g., a first scFv) and a second antigen binding domain (e.g., a second scFv) can each bind a TRBC polypeptide. For example, this document provides bispecific molecules that include at least two antigen binding domains where a first antigen binding domain (e.g., a first scFv) can bind a TRBC polypeptide and a second antigen binding domain (e.g., a second scFv) can bind a T cell co-receptor polypeptide (e.g., a CD3 polypeptide). This document also provides methods for treating T cell cancers. For example, one or more bispecific molecules provided herein (e.g., a composition containing one or more bispecific molecules provided herein) can be administered to a mammal having a T cell cancer to treat the mammal. In some cases, one or more bispecific molecules provided herein (e.g., one or more bispecific molecules including a first antigen binding domain and a second antigen binding domain that can each bind a TRBC polypeptide and, optionally, bispecific molecules including a first antigen binding domain that can bind a TRBC polypeptide and a second antigen binding domain that can bind a T cell co-receptor polypeptide such as a CD3 polypeptide) can be administered to a mammal to activate T cells within the mammal to target (e.g., target and destroy) T cells expressing a TRBC polypeptide that can be targeted by the bispecific molecule. For example, one or more bispecific molecules including a first antigen binding domain and a second antigen binding domain that can each bind a TRBC polypeptide can be administered to a mammal (e.g., a human) to activate T cells to target (e.g., target and destroy) T cells (e.g., cancerous T cells) expressing a TRBC polypeptide, and, optionally, bispecific molecules including a first antigen binding domain that can bind a TRBC polypeptide and a second antigen binding domain that can bind a T cell co-receptor polypeptide (e.g., a CD3 polypeptide) can be subsequently administered to the mammal to activate T cells to target (e.g., target and destroy) any remaining T cells (e.g., cancerous T cells) expressing the TRBC polypeptide.

Any appropriate mammal (e.g., a mammal having a clonal T cell expansion such as a T cell cancer) can be treated as described herein. For example, humans, non-human primates (e.g., monkeys), horses, bovine species, porcine species, dogs, cats, mice, and rats can be treated as described herein. In some cases, a human having a T cell cancer can be administered one or more bispecific molecules provided herein (e.g., bispecific molecules including a first antigen binding domain and a second antigen binding domain that can each bind a TRBC polypeptide and, optionally, bispecific molecules including a first antigen binding domain that can bind a TRBC polypeptide and a second antigen binding domain that can bind a T cell co-receptor polypeptide such as a CD3 polypeptide).

The materials and methods described herein can be used to treat a mammal (e.g., a human) having any type of T cell cancer. In some cases, a T cell cancer treated as described herein can include one or more solid tumors. In some cases, a T cell cancer treated as described herein can be a blood cancer. In some cases, a T cell cancer treated as described herein can be a primary cancer. In some cases, a T cell cancer treated as described herein can be a recurrent cancer. In some cases, a T cell cancer treated as described herein can be a metastatic cancer. In some cases, a T cell cancer treated as described herein can be a refractory cancer. In some cases, a T cell cancer treated as described herein can be a non-Hodgkin's lymphoma. Examples of T cell cancers that can be treated as described herein include, without limitation, acute lymphoblastic leukemia (ALL), peripheral T cell lymphomas (PTCL), angioimmunoblastic T cell lymphomas (AITL), T cell prolymphocytic leukemia (T-PLL), adult T cell leukemia/lymphoma (ATLL), enteropathy-associated T-cell lymphoma (EATL), monomorphic epitheliotropic intestinal T-cell lymphoma (MEITL), follicular T-cell lymphoma (FTCL), nodal peripheral T-cell lymphoma (nodal PTCL), cutaneous T cell lymphomas (CTCL), anaplastic large cell lymphoma (ALCL), T-cell large granular lymphocytic leukemia (T-LGL), extra nodal NK/T-Cell lymphoma (NKTL), and hepatosplenic T-cell lymphoma.

In some cases, the materials and methods provided herein can be used to reduce or eliminate the number of cancer cells present within a mammal (e.g., a human) having a T cell cancer. For example, a mammal in need thereof (e.g., a mammal having a T cell cancer) can be administered one or more bispecific molecules provided herein (e.g., one or more bispecific molecules including a first antigen binding domain and a second antigen binding domain that can each bind a TRBC polypeptide and, optionally, one or more bispecific molecules including a first antigen binding domain that can bind a TRBC polypeptide and a second antigen binding domain that can bind a T cell co-receptor polypeptide such as a CD3 polypeptide) to reduce or eliminate the number of cancer cells present within the mammal. For example, the materials and methods described herein can be used to reduce the number of cancer cells present within a mammal having cancer by, for example, 10, 20, 30, 40, 50, 60, 70, 80, 90, 95, or more percent. For example, the materials and methods described herein can be used to reduce the size (e.g., volume) of one or more tumors present within a mammal having cancer by, for example, 10, 20, 30, 40, 50, 60, 70, 80, 90, 95, or more percent. In some cases, the number of cancer cells present within a mammal being treated can be monitored. Any appropriate method can be used to determine whether or not the number of cancer cells present within a mammal is reduced. For example, imaging techniques can be used to assess the number of cancer cells present within a mammal.

In some cases, the materials and methods provided herein can be used to improve survival of a mammal (e.g., a human) having a T cell cancer. For example, a mammal in need thereof (e.g., a mammal having a T cell cancer) can be administered one or more bispecific molecules provided herein (e.g., one or more bispecific molecules including a first antigen binding domain and a second antigen binding domain that can each bind a TRBC polypeptide and, optionally, one or more bispecific molecules including a first antigen binding domain that can bind a TRBC polypeptide and a second antigen binding domain that can bind a T cell co-receptor polypeptide such as a CD3 polypeptide) to improve survival of the mammal. For example, the materials and methods described herein can be used to improve the survival of a mammal having cancer by, for example, 10, 20, 30, 40, 50, 60, 70, 80, 90, 95, or more percent. For example, the materials and methods described herein can be used to improve the survival of a mammal having cancer by, for example, at least 6 months (e.g., about 6 months, about 8 months, about 10 months, about 1 year, about 1.5 years, about 2 years, about 2.5 years, about 3 years, about 4 years, about 5 years, or more).

In some cases, when a mammal in need thereof (e.g., a mammal having a T cell cancer) is administered one or more bispecific molecules provided herein (e.g., bispecific molecules including a first antigen binding domain and a second antigen binding domain that can each bind a TRBC polypeptide and, optionally, bispecific molecules including a first antigen binding domain that can bind a TRBC polypeptide and a second antigen binding domain that can bind a T cell co-receptor polypeptide such as aCD3 polypeptide), the majority of normal T cells (e.g., a number of normal T cells sufficient to maintain adequate T cell immunity) within the mammal can be preserved. For example, the materials and methods described herein can be used to treat a mammal having a T cell cancer as described herein while preserving, for example, 20, 25, 30, 35, 40, 45, 50, 60, 70, 80, 90, 95, or more percent of normal (e.g., non-cancerous) T cells within the mammal. In some cases, from about 20 percent to about 75 percent (e.g., from about 20 percent to about 65 percent, from about 20 percent to about 55 percent, from about 20 percent to about 45 percent, from about 25 percent to about 75 percent, from about 35 percent to about 75 percent, from about 45 percent to about 75 percent, from about 55 percent to about 75 percent, from about 65 percent to about 75 percent, from about 35 percent to about 65 percent, from about 45 percent to about 55 percent, from about 30 percent to about 50 percent, from about 40 percent to about 60 percent, or from about 50 percent to about 70 percent) of normal (e.g., non-cancerous) T cells within a mammal can be preserved when the mammal is administered one or more bispecific molecules provided herein.

In some cases, the methods described herein also can include identifying a mammal as having a T cell cancer. Examples of methods for identifying a mammal as having a T cell cancer include, without limitation, physical examination, laboratory tests (e.g., blood and/or urine), biopsy, imaging tests (e.g., X-ray, PET/CT, Mill, and/or ultrasound), nuclear medicine scans (e.g., bone scans), endoscopy, and/or genetic tests. Once identified as having a T cell cancer, a mammal can be administered or instructed to self-administer one or more bispecific molecules provided herein (e.g., one or more bispecific molecules including a first antigen binding domain and a second antigen binding domain that can each bind a TRBC polypeptide and, optionally, one or more bispecific molecules including a first antigen binding domain that can bind a TRBC polypeptide and a second antigen binding domain that can bind a T cell co-receptor polypeptide such as a CD3 polypeptide).

Any appropriate bispecific molecule can be administered to a mammal (e.g., a human) as described herein. In some cases, a molecule provided herein can include at least two (e.g., two, three, four, five, six, seven, eight, nine, or ten) antigen binding domains. For example, a bispecific molecule can include at least two antigen binding domains where a first antigen binding domain (e.g., a first scFv) and a second antigen binding domain (e.g., a second scFv) can each bind a TRBC polypeptide. In some cases, a first antigen binding domain and a second antigen binding domain that can each bind a TRBC polypeptide can each bind the same epitope on the TRBC polypeptide. In some cases, a first antigen binding domain and a second antigen binding domain that can each bind a TRBC polypeptide can have different affinities toward the TRBC polypeptide(s). For example, a bispecific molecule can include at least two antigen binding domains where a first antigen binding domain (e.g., a first scFv) can bind a TRBC polypeptide with a lower affinity than the affinity with which a second antigen binding domain can bind the same TRBC polypeptide. In some cases, a first antigen binding domain and a second antigen binding domain that can each bind a TRBC polypeptide can bind different epitopes on the TRBC polypeptide. For example, a bispecific molecule can include at least two antigen binding domains where a first antigen binding domain (e.g., a first scFv) can bind a TRBC polypeptide and a second antigen binding domain that can bind a T cell co-receptor polypeptide (e.g., a CD3 polypeptide). Examples of molecules that can include at least two antigen binding domains (e.g., bispecific molecules) where a first antigen binding domain (e.g., a first scFv) and a second antigen binding domain (e.g., a second scFv) can each bind a TRBC polypeptide include, without limitation, single chain diabodies (scDbs), bispecific T cell engagers (BITEs), dual affinity retargeting molecules (DARTs), bivalent scFv-Fcs, and trivalent scFv-Fcs.

An antigen binding domain in a bispecific molecule provided herein (e.g., a bispecific molecule including a first antigen binding domain and a second antigen binding domain that can each bind a TRBC polypeptide and a bispecific molecule including a first antigen binding domain that can bind a TRBC polypeptide and a second antigen binding domain that can bind a T cell co-receptor polypeptide such as a CD3 polypeptide) can be any appropriate type of antigen binding domain. In some cases, an antigen binding domain that can be used in a bispecific molecule provided herein can include a variable region of an immunoglobulin light chain (a VL) and a variable region of an immunoglobulin heavy chain (VH). For example, an antigen binding domain that can be used in a bispecific molecule provided herein can include a first complementarity determining region (CDR) from an immunoglobulin light chain (a V_{L} CDR1), a second CDR from an immunoglobulin light chain (a V_{L} CDR2), and a third CDR an immunoglobulin light chain (a V_{L} CDR3), a first CDR from an immunoglobulin heavy chain (a V_{H} CDR1), a second CDR from an immunoglobulin heavy chain (a V_{H} CDR2), and a third CDR an immunoglobulin heavy chain (a V_{H} CDR2). Examples of antigen binding domains that can be used as a can be used as an antigen binding domain in a bispecific molecule provided herein include, without limitation, single-chain variable fragment (scFv), an antigen-binding fragment (Fab), a F(ab′)2 fragment, and biologically active fragments thereof (e.g., a fragment that retains the ability to bind the target molecule such as a TRBC polypeptide or a T cell co-receptor polypeptide such as a CD3 polypeptide). In some cases, an antigen binding domain that can be used as an antigen binding domain in a bispecific molecule provided herein can be a scFv. In some cases, the two antigen binding domains in a bispecific molecule provided herein can be the same type of antigen binding domains. For example each of the two antigen binding domains in a bispecific molecule provided herein can be a scFv. In some cases, the two antigen binding domains in a bispecific molecule provided herein can be different types of antigen binding domains.

In some cases, an antigen binding domain in a bispecific molecule provided herein (e.g., a bispecific molecule including a first antigen binding domain and a second antigen binding domain that can each bind a TRBC polypeptide and a bispecific molecule including a first antigen binding domain that can bind a TRBC polypeptide and a second antigen binding domain that can bind a T cell co-receptor polypeptide such as a CD3 polypeptide) can be a humanized antigen binding domain.

An antigen binding domain in a bispecific molecule provided herein (e.g., a bispecific molecule including a first antigen binding domain and a second antigen binding domain that can each bind a TRBC polypeptide and a bispecific molecule including a first antigen binding domain that can bind a TRBC polypeptide and a second antigen binding domain that can bind a T cell co-receptor polypeptide such as a CD3 polypeptide) that can bind a TRBC polypeptide can bind any appropriate TRBC polypeptide. Examples of TRBC polypeptides that can be targeted by an antigen binding domain in a bispecific molecule provided herein include, without limitation, TRBC1 polypeptides and TRBC2 polypeptides. In some cases, an antigen binding domain that can bind a TRBC polypeptide is specific for that TRBC polypeptide. For example, an antigen binding domain that can bind a TRBC polypeptide can bind to that TRBC polypeptide with an affinity having a dissociation constant (K_{D}) of from about 0.01 nM to about 400 nM (e.g., from about 0.01 nM to about 350 nM, from about 0.01 nM to about 300 nM, from about 0.01 nM to about 250 nM, from about 0.01 nM to about 200 nM, from about 0.01 nM to about 150 nM, from about 0.01 nM to about 100 nM, from about 0.01 nM to about 80 nM, from about 0.01 nM to about 50 nM, from about 0.01 nM to about 30 nM, from about 0.01 nM to about 10 nM, from about 0.01 nM to about 5 nM from about 0.01 nM to about 1 nM, from about 0.01 nM to about 0.5 nM, from about 0.4 nM to about 400 nM, from about 1 nM to about 400 nM, from about 5 nM to about 400 nM from about 10 nM to about 400 nM, from about 50 nM to about 400 nM, from about 100 nM to about 400 nM, from about 200 nM to about 400 nM, from about 300 nM to about 400 nM, from about 0.05 nM to about 200 nM, from about 0.1 nM to about 100 nM, from about 0.03 nM to about 50 nM, from about 0.04 nM to about 30 nM, from about 0.05 nM to about 10 nM, from about 0.1 nM to about 20 nM, from about 0.2 nM to about 30 nM, from about 0.3 nM to about 40 nM, from about 0.4 nM to about 50 nM, or from about 0.5 nM to about 100 nM). In some cases, an antigen binding domain that specifically binds a TRBC polypeptide does not bind (or does not substantially bind) a different TRBC polypeptide.

In some cases, an antigen binding domain that can be used in a bispecific molecule provided herein (e.g., a bispecific molecule including a first antigen binding domain and a second antigen binding domain that can each bind a TRBC polypeptide and a bispecific molecule including a first antigen binding domain that can bind a TRBC polypeptide and a second antigen binding domain that can bind a T cell co-receptor polypeptide such as a CD3 polypeptide) can bind to a TRBC1 polypeptide. For example, an antigen binding domain that can bind to a TRBC1 polypeptide can include each of the CDRs set forth below:

TABLE 1

| | Sequence | SEQ ID NO |
|---|---|---|
| V_{L} CDR1 | RSSQRLVHSNGNTYLH | 1 |
| V_{L} CDR2 | RVSNRFP | 2 |
| V_{L} CDR3 | SQSTHVPYT | 3 |
| V_{H} CDR1 | GYTFTGY | 4 |
| V_{H} CDR2 | NPYNDD | 5 |
| V_{H} CDR3 | GAGYNFDGAYRFFDF | 6 |

In some cases, an antigen binding domain that can be used in a bispecific molecule provided herein (e.g., a bispecific molecule including a first antigen binding domain and a second antigen binding domain that can each bind a TRBC polypeptide and a bispecific molecule including a first antigen binding domain that can bind a TRBC polypeptide and a second antigen binding domain that can bind a T cell co-receptor polypeptide such as a CD3 polypeptide) can have one or more CDRs that are not 100% identical to the CDRs set forth in Table 1, but retain the ability to bind to a TRBC1 polypeptide. For example, a CDR that includes one or more (e.g., one, two, three, four, five, or more) amino acid substitutions relative to a CDR set forth in Table 1 can be used in an antigen binding domain that can be used in a bispecific molecule provided herein. An amino acid substitution can be made, in some cases, by selecting a substitution that does not differ significantly in its effect on maintaining (a) the structure of the peptide backbone in the area of the substitution, (b) the charge or hydrophobicity of the molecule at particular sites, or (c) the bulk of the side chain. For example, naturally occurring residues can be divided into groups based on side-chain properties: (1) hydrophobic amino acids (methionine, alanine, valine, leucine, and isoleucine); (2) neutral hydrophilic amino acids (cysteine, serine, and threonine); (3) acidic amino acids (aspartic acid and glutamic acid); (4) basic amino acids (asparagine, glutamine, histidine, lysine, and arginine); (5) amino acids that influence chain orientation (glycine and proline); and (6) aromatic amino acids (tryptophan, tyrosine, and phenylalanine). Substitutions made within these groups can be considered conservative substitutions. Non-limiting examples of conservative substitutions that can be made within a CDR of an antigen binding domain that can be used in a bispecific molecule provided herein include, without limitation, substitution of valine for alanine, lysine for arginine, glutamine for asparagine, glutamic acid for aspartic acid, serine for cysteine, asparagine for glutamine, aspartic acid for glutamic acid, proline for glycine, arginine for histidine, leucine for isoleucine, isoleucine for leucine, arginine for lysine, leucine for methionine, leucine for phenyalanine, glycine for proline, threonine for serine, serine for threonine, tyrosine for tryptophan, phenylalanine for tyrosine, and/or leucine for valine.

In some cases, an antigen binding domain that can bind to a TRBC1 polypeptide can include a light chain having a V_{L} CDR1 including the amino acid sequence set forth in SEQ ID NO:1, a V_{L} CDR2 including the amino acid sequence set forth in SEQ ID NO:2, and a V_{L} CDR3 including the amino acid sequence set forth in SEQ ID NO:3. For example, an antigen binding domain that can bind to a TRBC1 polypeptide can include a light chain including the amino acid sequence set forth in SEQ ID NO:7. For example, an antigen binding domain that can bind to a TRBC1 polypeptide can include a light chain including the amino acid sequence set forth in SEQ ID NO:48. In some cases, an antigen binding domain that can bind to a TRBC1 polypeptide can include a heavy chain having a V_{H} CDR1 including the amino acid sequence set forth in SEQ ID NO:4, a V_{H} CDR2 including the amino acid sequence set forth in SEQ ID NO:5, and a V_{H} CDR3 including the amino acid sequence set forth in SEQ ID NO:6. For example, an antigen binding domain that can bind to a TRBC1 polypeptide can include a heavy chain including the amino acid sequence set forth in SEQ ID NO:8. For example, an antigen binding domain that can bind to a TRBC1 polypeptide can include a heavy chain including the amino acid sequence set forth in SEQ ID NO:49. In some cases, an antigen binding domain that can bind to a TRBC1 polypeptide can include a light chain having a V_{L} CDR1 including the amino acid sequence set forth in SEQ ID NO:1, a V_{L} CDR2 including the amino acid sequence set forth in SEQ ID NO:2, and a V_{L} CDR3 including the amino acid sequence set forth in SEQ ID NO:3, and can include a heavy chain having a V_{H} CDR1 including the amino acid sequence set forth in SEQ ID NO:4, a V_{H} CDR2 including the amino acid sequence set forth in SEQ ID NO:5, and a V_{H} CDR3 including the amino acid sequence set forth in SEQ ID NO:6. For example, an antigen binding domain that can bind to a TRBC1 polypeptide can include a light chain including the amino acid sequence set forth in SEQ ID NO:7 and can include a heavy chain including the amino acid sequence set forth in SEQ ID NO:8. For example, an antigen binding domain that can bind to a TRBC1 polypeptide can include a light chain including the amino acid sequence set forth in SEQ ID NO:48 and can include a heavy chain including the amino acid sequence set forth in SEQ ID NO:49.

In some cases, an antigen binding domain in a bispecific molecule provided herein (e.g., a bispecific molecule including a first antigen binding domain that can bind a TRBC polypeptide and a second antigen binding domain that can bind a T cell co-receptor polypeptide such as a CD3 polypeptide) that can bind a TRBC polypeptide can be as described elsewhere (see, e.g., Maciocia et al., Nat. Med., 23:1416-1423 (2017); and U.S. Patent Application Publication No. US 2017/0066827).

In cases where a bispecific molecule provided herein (e.g., a bispecific molecule including a first antigen binding domain and a second antigen binding domain that can each bind a TRBC polypeptide and a bispecific molecule including a first antigen binding domain that can bind a TRBC polypeptide and a second antigen binding domain that can bind a T cell co-receptor polypeptide such as a CD3 polypeptide) includes an antigen binding domain that can bind a T cell co-receptor polypeptide, the antigen binding domain that can bind a T cell co-receptor polypeptide can bind any appropriate T cell co-receptor polypeptide. Examples of T cell co-receptor polypeptides that can be targeted by a second antigen binding domain in a bispecific molecule provided herein include, without limitation, CD3 polypeptides such as CD3γ polypeptides, CD3δ polypeptides, and CD3ε polypeptides.

In some cases where a bispecific molecule provided herein includes an antigen binding domain that can bind a T cell co-receptor polypeptide, the bispecific molecule can bind to a CD3 polypeptide. For example, an antigen binding domain that can bind to a CD3 polypeptide can include one of each of the CDRs set forth below:

TABLE 2

| | Sequence | SEQ ID NO |
|---|---|---|
| V_{L} CDR1 | RASQDIRNYLN | 9 |
| V_{L} CDR1 | RASSSVSYMN | 19 |
| V_{L} CDR1 | SASSSVSYMN | 20 |
| V_{L} CDR1 | RSSTGAVTTSNYAN | 21 |
| V_{L} CDR1 | RASQSVSYMN | 22 |
| V_{L} CDR2 | (Y)YTSRLHS | 10 |
| | (with the first | |
| | Y being optional) | |
| V_{L} CDR2 | DTSKVAS | 23 |
| V_{L} CDR2 | DTSKLAS | 24 |
| V_{L} CDR2 | GTNKRAP | 25 |
| V_{L} CDR3 | QQGNTLPWT | 11 |
| V_{L} CDR3 | QQWSSNPLT | 26 |
| V_{L} CDR3 | QQWSSNPFT | 27 |
| V_{L} CDR3 | ALWYSNLWV | 28 |
| V_{H} CDR1 | GYTMN | 12 |
| V_{H} CDR1 | RYTMH | 29 |
| V_{H} CDR1 | TYAMN | 30 |
| V_{H} CDR2 | LINPYKGVSTYNQKFKD | 13 |
| V_{H} CDR2 | YINPSRGYTNYNQKFK | 31 |
| V_{H} CDR2 | RIRSKYNNYATYYADSVKD | 32 |
| V_{H} CDR2 | YINPSRGYTNYADSVKG | 33 |
| V_{H} CDR3 | SGYYGDSDWYFDV | 14 |
| V_{H} CDR3 | YYDDHYCLDY | 34 |
| V_{H} CDR3 | HGNFGNSYVSWFAY | 35 |

In some cases, an antigen binding domain that can be used in a bispecific molecule provided herein (e.g., a bispecific molecule including a first antigen binding domain and a second antigen binding domain that can each bind a TRBC polypeptide and a bispecific molecule including a first antigen binding domain that can bind a TRBC polypeptide and a second antigen binding domain that can bind a T cell co-receptor polypeptide such as a CD3 polypeptide) can have one or more CDRs that are not 100% identical to the CDRs set forth in Table 2, but retain the ability to bind to a TRBC1 polypeptide. For example, a CDR that includes one or more (e.g., one, two, three, four, five, or more) amino acid substitutions relative to a CDR set forth in Table 2 can be used in an antigen binding domain that can be used in a bispecific molecule provided herein. An amino acid substitution can be made, in some cases, by selecting a substitution that does not differ significantly in its effect on maintaining (a) the structure of the peptide backbone in the area of the substitution, (b) the charge or hydrophobicity of the molecule at particular sites, or (c) the bulk of the side chain. For example, naturally occurring residues can be divided into groups based on side-chain properties: (1) hydrophobic amino acids (methionine, alanine, valine, leucine, and isoleucine); (2) neutral hydrophilic amino acids (cysteine, serine, and threonine); (3) acidic amino acids (aspartic acid and glutamic acid); (4) basic amino acids (asparagine, glutamine, histidine, lysine, and arginine); (5) amino acids that influence chain orientation (glycine and proline); and (6) aromatic amino acids (tryptophan, tyrosine, and phenylalanine). Substitutions made within these groups can be considered conservative substitutions. Non-limiting examples of conservative substitutions that can be made within a CDR of an antigen binding domain that can be used in a bispecific molecule provided herein include, without limitation, substitution of valine for alanine, lysine for arginine, glutamine for asparagine, glutamic acid for aspartic acid, serine for cysteine, asparagine for glutamine, aspartic acid for glutamic acid, proline for glycine, arginine for histidine, leucine for isoleucine, isoleucine for leucine, arginine for lysine, leucine for methionine, leucine for phenyalanine, glycine for proline, threonine for serine, serine for threonine, tyrosine for tryptophan, phenylalanine for tyrosine, and/or leucine for valine.

In some cases, an antigen binding domain that can bind to a CD3 polypeptide can include a light chain having a V_{L} CDR1 including the amino acid sequence set forth in SEQ ID NO:9, a V_{L} CDR2 including the amino acid sequence set forth in SEQ ID NO:10, and a V_{L} CDR3 including the amino acid sequence set forth in SEQ ID NO:11. For example, an antigen binding domain that can bind to a CD3 polypeptide can include a light chain including the amino acid sequence set forth in SEQ ID NO:15. For example, an antigen binding domain that can bind to a CD3 polypeptide can include a light chain including the amino acid sequence set forth in SEQ ID NO:17.

In some cases, an antigen binding domain that can bind to a CD3 polypeptide can include a light chain having a V_{L} CDR1 including the amino acid sequence set forth in SEQ ID NO:19, a V_{L} CDR2 including the amino acid sequence set forth in SEQ ID NO:23, and a V_{L} CDR3 including the amino acid sequence set forth in SEQ ID NO:26. For example, an antigen binding domain that can bind to a CD3 polypeptide can include a light chain including the amino acid sequence set forth in SEQ ID NO:36.

In some cases, an antigen binding domain that can bind to a CD3 polypeptide can include a light chain having a V_{L} CDR1 including the amino acid sequence set forth in SEQ ID NO:20, a V_{L} CDR2 including the amino acid sequence set forth in SEQ ID NO:24, and a V_{L} CDR3 including the amino acid sequence set forth in SEQ ID NO:27. For example, an antigen binding domain that can bind to a CD3 polypeptide can include a light chain including the amino acid sequence set forth in SEQ ID NO:38.

In some cases, an antigen binding domain that can bind to a CD3 polypeptide can include a light chain having a V_{L} CDR1 including the amino acid sequence set forth in SEQ ID NO:21, a V_{L} CDR2 including the amino acid sequence set forth in SEQ ID NO:25, and a V_{L} CDR3 including the amino acid sequence set forth in SEQ ID NO:28. For example, an antigen binding domain that can bind to a CD3 polypeptide can include a light chain including the amino acid sequence set forth in SEQ ID NO:40.

In some cases, an antigen binding domain that can bind to a CD3 polypeptide can include a light chain having a V_{L} CDR1 including the amino acid sequence set forth in SEQ ID NO:22, a V_{L} CDR2 including the amino acid sequence set forth in SEQ ID NO:23, and a V_{L} CDR3 including the amino acid sequence set forth in SEQ ID NO:26. For example, an antigen binding domain that can bind to a CD3 polypeptide can include a light chain including the amino acid sequence set forth in SEQ ID NO:42.

In some cases, an antigen binding domain that can bind to a CD3 polypeptide can include a heavy chain having a V_{H} CDR1 including the amino acid sequence set forth in SEQ ID NO:12, a V_{H} CDR2 including the amino acid sequence set forth in SEQ ID NO:13, and a V_{H} CDR3 including the amino acid sequence set forth in SEQ ID NO:14. For example, an antigen binding domain that can bind to a CD3 polypeptide can include a heavy chain including the amino acid sequence set forth in SEQ ID NO:16. For example, an antigen binding domain that can bind to a CD3 polypeptide can include a heavy chain including the amino acid sequence set forth in SEQ ID NO:18.

In some cases, an antigen binding domain that can bind to a CD3 polypeptide can include a heavy chain having a V_{H} CDR1 including the amino acid sequence set forth in SEQ ID NO:29, a V_{H} CDR2 including the amino acid sequence set forth in SEQ ID NO:31, and a V_{H} CDR3 including the amino acid sequence set forth in SEQ ID NO:34. For example, an antigen binding domain that can bind to a CD3 polypeptide can include a heavy chain including the amino acid sequence set forth in SEQ ID NO:37.

In some cases, an antigen binding domain that can bind to a CD3 polypeptide can include a heavy chain having a V_{H} CDR1 including the amino acid sequence set forth in SEQ ID NO:29, a V_{H} CDR2 including the amino acid sequence set forth in SEQ ID NO:31, and a V_{H} CDR3 including the amino acid sequence set forth in SEQ ID NO:34. For example, an antigen binding domain that can bind to a CD3 polypeptide can include a heavy chain including the amino acid sequence set forth in SEQ ID NO:39.

In some cases, an antigen binding domain that can bind to a CD3 polypeptide can include a heavy chain having a V_{H} CDR1 including the amino acid sequence set forth in SEQ ID NO:30, a V_{H} CDR2 including the amino acid sequence set forth in SEQ ID NO:32, and a V_{H} CDR3 including the amino acid sequence set forth in SEQ ID NO:35. For example, an antigen binding domain that can bind to a CD3 polypeptide can include a heavy chain including the amino acid sequence set forth in SEQ ID NO:41.

In some cases, an antigen binding domain that can bind to a CD3 polypeptide can include a heavy chain having a V_{H} CDR1 including the amino acid sequence set forth in SEQ ID NO:29, a V_{H} CDR2 including the amino acid sequence set forth in SEQ ID NO:33, and a V_{H} CDR3 including the amino acid sequence set forth in SEQ ID NO:34. For example, an antigen binding domain that can bind to a CD3 polypeptide can include a heavy chain including the amino acid sequence set forth in SEQ ID NO:43.

In some cases, an antigen binding domain that can bind to a CD3 polypeptide can include a light chain having a V_{L} CDR1 including the amino acid sequence set forth in SEQ ID NO:9, a V_{L} CDR2 including the amino acid sequence set forth in SEQ ID NO:10, and a V_{L} CDR3 including the amino acid sequence set forth in SEQ ID NO:11, and can include a heavy chain having a V_{H} CDR1 including the amino acid sequence set forth in SEQ ID NO:12, a V_{H} CDR2 including the amino acid sequence set forth in SEQ ID NO:13, and a V_{H} CDR3 including the amino acid sequence set forth in SEQ ID NO:14. For example, an antigen binding domain that can bind to a CD3 polypeptide can include a light chain including the amino acid sequence set forth in SEQ ID NO:15 and can include a heavy chain including the amino acid sequence set forth in SEQ ID NO:16. For example, an antigen binding domain that can bind to a CD3 polypeptide can include a light chain including the amino acid sequence set forth in SEQ ID NO:17 and can include a heavy chain including the amino acid sequence set forth in SEQ ID NO:18.

In some cases, an antigen binding domain that can bind to a CD3 polypeptide can include a light chain having a VL CDR1 including the amino acid sequence set forth in SEQ ID NO:22, a VL CDR2 including the amino acid sequence set forth in SEQ ID NO:23, and a VL CDR3 including the amino acid sequence set forth in SEQ ID NO:26, and can include a heavy chain having a VH CDR1 including the amino acid sequence set forth in SEQ ID NO:29, a VH CDR2 including the amino acid sequence set forth in SEQ ID NO:33, and a VH CDR3 including the amino acid sequence set forth in SEQ ID NO:34. For example, an antigen binding domain that can bind to a CD3 polypeptide can include a light chain including the amino acid sequence set forth in SEQ ID NO:42 and can include a heavy chain including the amino acid sequence set forth in SEQ ID NO:43.

In some cases, an antigen binding domain that can bind to a CD3 polypeptide can include a light chain having a VL CDR1 including the amino acid sequence set forth in SEQ ID NO:20, a VL CDR2 including the amino acid sequence set forth in SEQ ID NO:24, and a VL CDR3 including the amino acid sequence set forth in SEQ ID NO:27, and can include a heavy chain having a VH CDR1 including the amino acid sequence set forth in SEQ ID NO:29, a VH CDR2 including the amino acid sequence set forth in SEQ ID NO:31, and a VH CDR3 including the amino acid sequence set forth in SEQ ID NO:34. For example, an antigen binding domain that can bind to a CD3 polypeptide can include a light chain including the amino acid sequence set forth in SEQ ID NO:38 and can include a heavy chain including the amino acid sequence set forth in SEQ ID NO:39.

In some cases, an antigen binding domain that can bind to a CD3 polypeptide can include a light chain having a VL CDR1 including the amino acid sequence set forth in SEQ ID NO:21, a VL CDR2 including the amino acid sequence set forth in SEQ ID NO:25, and a VL CDR3 including the amino acid sequence set forth in SEQ ID NO:28, and can include a heavy chain having a VH CDR1 including the amino acid sequence set forth in SEQ ID NO:30, a VH CDR2 including the amino acid sequence set forth in SEQ ID NO:32, and a VH CDR3 including the amino acid sequence set forth in SEQ ID NO:35. For example, an antigen binding domain that can bind to a CD3 polypeptide can include a light chain including the amino acid sequence set forth in SEQ ID NO:40 and can include a heavy chain including the amino acid sequence set forth in SEQ ID NO:41.

In some cases, an antigen binding domain that can bind to a CD3 polypeptide can include a light chain having a V_{L} CDR1 including the amino acid sequence set forth in SEQ ID NO:22, a V_{L} CDR2 including the amino acid sequence set forth in SEQ ID NO:23, and a V_{L} CDR3 including the amino acid sequence set forth in SEQ ID NO:26, and can include a heavy chain having a VH CDR1 including the amino acid sequence set forth in SEQ ID NO:29, a VH CDR2 including the amino acid sequence set forth in SEQ ID NO:33, and a VH CDR3 including the amino acid sequence set forth in SEQ ID NO:34. For example, an antigen binding domain that can bind to a CD3 polypeptide can include a light chain including the amino acid sequence set forth in SEQ ID NO:42 and can include a heavy chain including the amino acid sequence set forth in SEQ ID NO:43.

In cases where a bispecific molecule provided herein (e.g., a bispecific molecule including a first antigen binding domain and a second antigen binding domain that can each bind a TRBC polypeptide and a bispecific molecule including a first antigen binding domain that can bind a TRBC polypeptide and a second antigen binding domain that can bind a T cell co-receptor polypeptide such as a CD3 polypeptide) includes an antigen binding domain that can bind a T cell co-receptor polypeptide, the antigen binding domain that can bind a T cell co-receptor polypeptide can be as described elsewhere (see, e.g., Zhu et al., Journal of Immunology, 155:1903-1910 (1995); Junttila et al., Cancer Research, 74:5561-5571 (2014); and Rodrigues et al., Int. J. Cancer. Suppl., 7:45-50 (1992)).

In some cases, a first antigen binding domain and a second antigen binding domain in a bispecific molecule provided herein (e.g., a bispecific molecule including a first antigen binding domain and a second antigen binding domain that can each bind a TRBC polypeptide and a bispecific molecule including a first antigen binding domain that can bind a TRBC polypeptide and a second antigen binding domain that can bind a T cell co-receptor polypeptide such as a CD3 polypeptide) can be connected via a linker (e.g., a polypeptide linker). A linker can include any appropriate number of amino acids. For example, a linker can include from about 5 amino acids to about 20 amino acids (e.g., from about 5 amino acids to about 20 amino acids, from about 5 amino acids to about 17 amino acids, from about 5 amino acids to about 15 amino acids, from about 5 amino acids to about 12 amino acids, from about 5 amino acids to about 10 amino acids, from about 5 amino acids to about 8 amino acids, from about 7 amino acids to about 20 amino acids, from about 10 amino acids to about 20 amino acids, from about 13 amino acids to about 20 amino acids, from about 15 amino acids to about 18 amino acids, from about 7 amino acids to about 18 amino acids, from about 10 amino acids to about 15 amino acids, from about 7 amino acids to about 12 amino acids, from about 10 amino acids to about 16 amino acids, or from about 12 amino acids to about 18 amino acids). In some cases, a linker can alter the flexibility of the bispecific molecule. In some cases, a linker can alter the solubility of the bispecific molecule. A linker can include any appropriate amino acids. In some cases, a linker can be a glycine-rich linker. In some cases, a linker can be serine and/or threonine-rich linker. A linker can connect the first antigen binding domain and the second antigen binding domain in a bispecific molecule provided herein in any order. For example, a linker can connect the N-terminus of a first antigen binding domain in a bispecific molecule provided herein with the C-terminus of the second antigen binding domain in a bispecific molecule, or vice versa. Examples of linkers that can be used to connect a first antigen binding domain and a second antigen binding domain in a bispecific molecule provided herein include, without limitation, a GGGGS linker (SEQ ID NO:44), a (GGGGS)₃ linker (SEQ ID NO:45), and VEGGSGGSGGSGGSGGVD (SEQ ID NO:46). In some cases, a linker described herein can also be used to connect a VH and a VL of an antigen binding domain described herein.

In some cases, a bispecific molecule provided herein (e.g., a bispecific molecule including a first antigen binding domain and a second antigen binding domain that can each bind a TRBC polypeptide and a bispecific molecule including a first antigen binding domain that can bind a TRBC polypeptide and a second antigen binding domain that can bind a T cell co-receptor polypeptide such as a CD3 polypeptide) can include one or more additional molecules (e.g., one or more additional polypeptides and/or one or more additional nanoparticles). In some cases, an additional molecule can alter (e.g., improve) the stability of the bispecific molecule. For example, an additional molecule can increase a half-life of a bispecific molecule provided herein (e.g., following administration to a mammal such as a human having a T-cell cancer). In some cases, an additional molecule can be used to detect (e.g., visualize) the bispecific molecule (e.g., following administration to a mammal such as a human having a T-cell cancer). In some cases, an additional molecule can be used to bind (e.g., to isolate and/or purify) the bispecific molecule. When an additional molecule is a polypeptide, the polypeptide can include any appropriate amino acids. When an additional molecule is a polypeptide, the polypeptide can include any appropriate number of amino acids. For example, an additional polypeptide can include from about 2 amino acids to about 10 amino acids (e.g., from about 2 amino acids to about 8 amino acids, from about 2 amino acids to about 6 amino acids, from about 4 amino acids to about 10 amino acids, from about 6 amino acids to about 10 amino acids, or from about 4 amino acids to about 8 amino acids). In some cases, an additional polypeptide can be a poly-histidine polypeptide (e.g., a poly-histidine tail or a poly-histidine tag). An additional molecule can be at any appropriate location within a bispecific molecule provided herein. For example, an additional molecule can be on the N-terminus or the bispecific molecule, on the C-terminus of the bispecific molecule, or on both the N-terminus and the C-terminus of the bispecific molecule. Examples of additional molecules that can be included in a bispecific molecule provided herein include, without limitation, a binding domain that can target albumin, an albumin polypeptide (or a fragment thereof), a crystallizable fragment (Fc) region, and a poly-histidine polypeptide such as a poly-histidine polypeptide including the amino acid sequence HHHHHH (SEQ ID NO:47). In some cases, an additional molecule that can be included in a bispecific molecule provided herein can be as described elsewhere (see, e.g., Davé et al., MAbs, 8(7):1319-1335 (2016); Müller et al., J. Biol. Chem., 282(17):12650-12660 (2007); and Liu et al., Front. Immunol., 8:38 (2017)).

In some cases, one or more bispecific molecules provided herein (e.g., bispecific molecules including a first antigen binding domain and a second antigen binding domain that can each bind a TRBC polypeptide or bispecific molecules including a first antigen binding domain that can bind a TRBC polypeptide and a second antigen binding domain that can bind a T cell co-receptor polypeptide such as a CD3 polypeptide) can be formulated into a composition (e.g., a pharmaceutical composition) for administration to a mammal (e.g., a human). For example, one or more bispecific molecules provided herein can be formulated into a pharmaceutically acceptable composition for administration to a mammal (e.g., a human) having a T cell cancer. In some cases, one or more bispecific molecules provided herein can be formulated together with one or more pharmaceutically acceptable carriers (additives), excipients, preservatives, stabilizers, and/or diluents. Examples of pharmaceutically acceptable carriers, excipients, preservatives, stabilizers, and diluents that can be used in a composition described herein include, without limitation, sucrose, lactose, starch (e.g., starch glycolate), cellulose, cellulose derivatives (e.g., modified celluloses such as microcrystalline cellulose and cellulose ethers like hydroxypropyl cellulose (HPC) and cellulose ether hydroxypropyl methylcellulose (HPMC)), xylitol, sorbitol, mannitol, gelatin, polymers (e.g., polyvinylpyrrolidone (PVP), polyethylene glycol (PEG), crosslinked polyvinylpyrrolidone (crospovidone), carboxymethyl cellulose, polyethylene-polyoxypropylene-block polymers, and crosslinked sodium carboxymethyl cellulose (croscarmellose sodium)), titanium oxide, azo dyes, silica gel, fumed silica, talc, magnesium carbonate, vegetable stearin, magnesium stearate, aluminum stearate, stearic acid, antioxidants (e.g., vitamin A, vitamin E, vitamin C, retinyl palmitate, and selenium), citric acid, sodium citrate, parabens (e.g., methyl paraben and propyl paraben), petrolatum, dimethyl sulfoxide, mineral oil, serum proteins (e.g., human serum albumin), glycine, sorbic acid, potassium sorbate, water, salts or electrolytes (e.g., saline, protamine sulfate, disodium hydrogen phosphate, potassium hydrogen phosphate, zinc salts, and sodium chloride such as bacteriostatic 0.9% sodium chloride), colloidal silica, magnesium trisilicate, polyacrylates, waxes, wool fat, and lecithin.

A composition (e.g., a pharmaceutical composition) containing one or more bispecific molecules provided herein (e.g., bispecific molecules including a first antigen binding domain and a second antigen binding domain that can each bind a TRBC polypeptide or bispecific molecules including a first antigen binding domain that can bind a TRBC polypeptide and a second antigen binding domain that can bind a T cell co-receptor polypeptide such as a CD3 polypeptide) can be formulated into any appropriate dosage form. Examples of dosage forms include solid or liquid forms including, without limitation, pills, capsules, tablets, gels, liquids, suspensions, solutions (e.g., sterile solutions), sustained-release formulations, and delayed-release formulations.

A composition (e.g., a pharmaceutical composition) containing one or more bispecific molecules provided herein (e.g., bispecific molecules including a first antigen binding domain and a second antigen binding domain that can each bind a TRBC polypeptide or bispecific molecules including a first antigen binding domain that can bind a TRBC polypeptide and a second antigen binding domain that can bind a T cell co-receptor polypeptide such as a CD3 polypeptide) can be designed for oral or parenteral (e.g., topical, subcutaneous, intravenous, intraperitoneal, intrathecal, and intraventricular) administration. When being administered orally, a composition can be in the form of a pill, tablet, or capsule. Compositions suitable for parenteral administration include aqueous and non-aqueous sterile injection solutions that can contain anti-oxidants, buffers, bacteriostats, and solutes which render the formulation isotonic with the blood of the intended recipient; and aqueous and non-aqueous sterile suspensions which may include suspending agents and thickening agents. The formulations can be presented in unit-dose or multi-dose containers, for example, sealed ampules and vials, and may be stored in a freeze dried (lyophilized) condition requiring only the addition of the sterile liquid carrier, for example water for injections, immediately prior to use. Extemporaneous injection solutions and suspensions may be prepared from sterile powders, granules, and tablets.

A composition (e.g., a pharmaceutical composition) containing one or more bispecific molecules provided herein (e.g., bispecific molecules including a first antigen binding domain and a second antigen binding domain that can each bind a TRBC polypeptide or bispecific molecules including a first antigen binding domain that can bind a TRBC polypeptide and a second antigen binding domain that can bind a T cell co-receptor polypeptide such as a CD3 polypeptide) can be administered locally or systemically. For example, a composition containing one or more bispecific molecules provided herein can be administered locally by a sentinel node injection to one or more lymph nodes within a mammal (e.g., a human). For example, a composition containing one or more bispecific molecules provided herein can be administered locally by an intravenous injection to a mammal (e.g., a human). For example, a composition containing one or more bispecific molecules provided herein can be administered locally by an intraperitoneal injection to a mammal (e.g., a human).

An effective amount (e.g., effective dose) of one or more bispecific molecules provided herein (e.g., bispecific molecules including a first antigen binding domain and a second antigen binding domain that can each bind a TRBC polypeptide or bispecific molecules including a first antigen binding domain that can bind a TRBC polypeptide and a second antigen binding domain that can bind a T cell co-receptor polypeptide such as a CD3 polypeptide) can vary depending on the severity of the T cell cancer, the route of administration, the age and general health condition of the subject, excipient usage, the possibility of co-usage with other therapeutic treatments such as use of other agents, and/or the judgment of the treating physician.

An effective amount of a composition (e.g., a pharmaceutical composition) containing one or more bispecific molecules provided herein (e.g., bispecific molecules including a first antigen binding domain and a second antigen binding domain that can each bind a TRBC polypeptide or bispecific molecules including a first antigen binding domain that can bind a TRBC polypeptide and a second antigen binding domain that can bind a T cell co-receptor polypeptide such as a CD3 polypeptide) can be any amount that can treat a mammal (e.g., a human) having a T cell cancer with a reasonable therapeutic index and/or without producing significant toxicity to the mammal. An effective amount of one or more bispecific molecules provided herein can be any appropriate amount. The effective amount can remain constant or can be adjusted as a sliding scale or variable dose depending on the mammal's response to treatment. Various factors can influence the actual effective amount used for a particular application. For example, the frequency of administration, duration of treatment, use of multiple treatment agents, route of administration, and severity of the condition (e.g., a T cell cancer) may require an increase or decrease in the actual effective amount administered.

The frequency of administration of a composition (e.g., a pharmaceutical composition) containing one or more bispecific molecules provided herein (e.g., bispecific molecules including a first antigen binding domain and a second antigen binding domain that can each bind a TRBC polypeptide or bispecific molecules including a first antigen binding domain that can bind a TRBC polypeptide and a second antigen binding domain that can bind a T cell co-receptor polypeptide such as a CD3 polypeptide) can be any frequency that can treat a mammal (e.g., a human) having a T cell cancer with a reasonable therapeutic index and/or without producing significant toxicity to the mammal. For example, the frequency of administration can be from about two times a day to about once a week, once every two weeks, once every 3 weeks, once every 4 weeks, once every 5 weeks, once every 6 weeks, once every 2 months, once every 3 months, or once every 4 months. In some cases, an administration can be a continuous administration (e.g., a continuous infusion). The frequency of administration can remain constant or can be variable during the duration of treatment. A course of treatment with a composition containing one or more bispecific molecules provided herein can include rest periods. As with the effective amount, various factors can influence the actual frequency of administration used for a particular application. For example, the effective amount, duration of treatment, use of multiple treatment agents, route of administration, and severity of the condition (e.g., a T cell cancer) may require an increase or decrease in administration frequency.

An effective duration for administering a composition (e.g., a pharmaceutical composition) containing one or more bispecific molecules provided herein (e.g., bispecific molecules including a first antigen binding domain and a second antigen binding domain that can each bind a TRBC polypeptide or bispecific molecules including a first antigen binding domain that can bind a TRBC polypeptide and a second antigen binding domain that can bind a T cell co-receptor polypeptide such as a CD3 polypeptide) can be any duration that treat a mammal (e.g., a human) having a T cell cancer with a reasonable therapeutic index and/or without producing significant toxicity to the mammal. For example, the effective duration can vary from several days to several weeks, months, or years. In some cases, the effective duration for the treatment of a mammal can range in duration from about one month to about 10 years. Multiple factors can influence the actual effective duration used for a particular treatment. For example, an effective duration can vary with the frequency of administration, effective amount, use of multiple treatment agents, route of administration, and severity of the condition (e.g., a T cell cancer) being treated.

In some cases, the methods and materials described herein can include administering one or more bispecific molecules including a first antigen binding domain and a second antigen binding domain that can each bind a TRBC polypeptide. For example, a mammal in need thereof (e.g., a mammal having a T cell cancer) can be administered one or more bispecific molecules including a first antigen binding domain and a second antigen binding domain that can each bind a TRBC polypeptide but is not administered any bispecific molecules including a first antigen binding domain that can bind a TRBC polypeptide and a second antigen binding domain that can bind a T cell co-receptor polypeptide (e.g., a CD3 polypeptide).

In some cases, the methods and materials described herein can include administering both one or more bispecific molecules including a first antigen binding domain and a second antigen binding domain that can each bind a TRBC polypeptide and one or more bispecific molecules including a first antigen binding domain that can bind a TRBC polypeptide and a second antigen binding domain that can bind a T cell co-receptor polypeptide (e.g., a CD3 polypeptide). For example, a mammal in need thereof (e.g., a mammal having a T cell cancer) can be administered one or more bispecific molecules including a first antigen binding domain and a second antigen binding domain that can each bind a TRBC polypeptide, and can subsequently be administered one or more bispecific molecules including a first antigen binding domain that can bind a TRBC polypeptide and a second antigen binding domain that can bind a T cell co-receptor polypeptide (e.g., a CD3 polypeptide). In some cases, methods that include administering both one or more bispecific molecules including a first antigen binding domain and a second antigen binding domain that can each bind a TRBC polypeptide and bispecific molecules including a first antigen binding domain that can bind a TRBC polypeptide and a second antigen binding domain that can bind a T cell co-receptor polypeptide (e.g., a CD3 polypeptide) can include a rest period between administering the one or more bispecific molecules including a first antigen binding domain and a second antigen binding domain that can each bind a TRBC polypeptide and administering the one or more bispecific molecules including a first antigen binding domain that can bind a TRBC polypeptide and a second antigen binding domain that can bind a T cell co-receptor polypeptide (e.g., a CD3 polypeptide). In some cases, one or more bispecific molecules including a first antigen binding domain and a second antigen binding domain that can each bind a TRBC polypeptide can be administered to a mammal having a T-cell cancer, and one or more bispecific molecules including a first antigen binding domain that can bind a TRBC polypeptide and a second antigen binding domain that can bind a T cell co-receptor polypeptide (e.g., a CD3 polypeptide) can be administered to the same mammal if/when any relapse of the T-cell cancer is observed in the mammal. For example, one or more bispecific molecules including a first antigen binding domain and a second antigen binding domain that can each bind a TRBC polypeptide can be administered to a mammal having a T-cell cancer, and one or more bispecific molecules including a first antigen binding domain that can bind a TRBC polypeptide and a second antigen binding domain that can bind a T cell co-receptor polypeptide (e.g., a CD3 polypeptide) can be administered to the same mammal from immediately after a relapse of the T-cell cancer is observed in the mammal to years after a relapse of the T-cell cancer is observed in the mammal. For example, administration of one or more bispecific molecules including a first antigen binding domain and a second antigen binding domain that can each bind a TRBC polypeptide to a mammal and an administration of one or more bispecific molecules including a first antigen binding domain that can bind a TRBC polypeptide and a second antigen binding domain that can bind a T cell co-receptor polypeptide (e.g., a CD3 polypeptide) to the same mammal can be administered with from about 30 days to about 10 years apart separating the administrations.

In some cases, one or more bispecific molecules provided herein (e.g., bispecific molecules including a first antigen binding domain and a second antigen binding domain that can each bind a TRBC polypeptide and, optionally, bispecific molecules including a first antigen binding domain that can bind a TRBC polypeptide and a second antigen binding domain that can bind a T cell co-receptor polypeptide such as a CD3 polypeptide) can be used as the sole active agent(s) to treat a mammal (e.g., a human) having a T cell cancer. For example, one or more bispecific molecules including a first antigen binding domain and a second antigen binding domain that can each bind a TRBC polypeptide can be used as the sole active agent to treat a mammal (e.g., a human) having a T cells cancer. For example, one or more bispecific molecules including a first antigen binding domain and a second antigen binding domain that can each bind a TRBC polypeptide and bispecific molecules including a first antigen binding domain that can bind a TRBC polypeptide and a second antigen binding domain that can bind a T cell co-receptor polypeptide (e.g., a CD3 polypeptide) can be used as the sole active agents to treat a mammal (e.g., a human) having a T cell cancer.

In some cases, the methods and materials described herein can include one or more (e.g., one, two, three, four, five or more) additional therapeutic agents used to treat a mammal (e.g., a human) having a T cell cancer. For example, a mammal in need thereof (e.g., a mammal having a T cell cancer) can be administered one or more bispecific molecules provided herein (e.g., one or more bispecific molecules including a first antigen binding domain and a second antigen binding domain that can each bind a TRBC polypeptide and, optionally, one or more bispecific molecules including a first antigen binding domain that can bind a TRBC polypeptide and a second antigen binding domain that can bind a T cell co-receptor polypeptide such as a CD3 polypeptide) in combination with one or more anti-cancer agents (e.g., chemotherapeutic agents). In some cases, an anti-cancer agent can be an alkylating agent. In some cases, an anti-cancer agent can be a platinum compound. In some cases, an anti-cancer agent can be a taxane. In some cases, an anti-cancer agent can be a luteinizing-hormone-releasing hormone (LHRH) agonist. In some cases, an anti-cancer agent can be an anti-estrogen. In some cases, an anti-cancer agent can be an aromatase inhibitor. In some cases, an anti-cancer agent can be an angiogenesis inhibitor. In some cases, an anti-cancer agent can be a checkpoint inhibitor. In some cases, an anti-cancer agent can be an immunotherapeutic agent. In some cases, an anti-cancer agent can be a poly(ADP)-ribose polymerase (PARP) inhibitor. In some cases, an anti-cancer agent can be a cytotoxic T-lymphocyte-associated protein 4 (CTLA4) inhibitor. In some cases, an anti-cancer agent can be an inhibitor of PD/PD-L1 signaling. In some cases, an anti-cancer agent can target one or more epigenetic alterations (e.g., DNA methylation and histone modifications). Examples of anti-cancer agents include, without limitation, vincristine, prednisone, dexamethasone, busulfan, cisplatin, carboplatin, paclitaxel, docetaxel, nab-paclitaxel, altretamine, capecitabine, cyclophosphamide, etoposide (vp-16), gemcitabine, ifosfamide, irinotecan (cpt-11), liposomal doxorubicin, melphalan, pemetrexed, topotecan, vinorelbine, goserelin, leuprolide, tamoxifen, letrozole, anastrozole, exemestane, bevacizumab, olaparib, rucaparib, niraparib, nivolumab, pembrolizumab, durvalumab, atezolizumab, radioisotopes, monomethyl auristatin E (MMAE; e.g., vedotin), calicheamicins, deruxtecan, DM1, and any combinations thereof. In some cases, an anti-cancer agent can be as described elsewhere (see, e.g., Zhang et al., Clin. Epigenet., 12:169 (2020) at, for example, Table 3 and Table 5; and Ghione et al., Curr. Hematol. Malig. Rep., 13(6):494-506 (2018) at, for example, section II, section III, and Table 1). In some cases, the one or more additional therapeutic agents can be administered together with one or more bispecific molecules provided herein (e.g., in a single composition). For example, an antigen binding domain that can bind a TRBC polypeptide described herein can be conjugated to one or more anti-cancer agents (e.g., can be in the form of an ADC). In some cases, the one or more additional therapeutic agents can be administered independent of the one or more bispecific molecules provided herein. When the one or more additional therapeutic agents are administered independent of the one or more bispecific molecules provided herein, the one or more bispecific molecules provided herein can be administered first, and the one or more additional therapeutic agents administered second, or vice versa.

In some cases, the methods and materials described herein can include administering both one or more bispecific molecules including a first antigen binding domain and a second antigen binding domain that can each bind a TRBC polypeptide and one or more molecules including a first antigen binding domain that can bind a TRBC polypeptide that is conjugated to one or more anti-cancer agents. For example, a mammal in need thereof (e.g., a mammal having a T cell cancer) can be administered one or more bispecific molecules including a first antigen binding domain and a second antigen binding domain that can each bind a TRBC polypeptide, and can subsequently be administered one or more molecules including a first antigen binding domain that can bind a TRBC polypeptide that is conjugated to one or more anti-cancer agents. In some cases, methods that include administering both one or more bispecific molecules including a first antigen binding domain and a second antigen binding domain that can each bind a TRBC polypeptide and one or more molecules including a first antigen binding domain that can bind a TRBC polypeptide that is conjugated to one or more anti-cancer agents can include a rest period between administering the one or more bispecific molecules including a first antigen binding domain and a second antigen binding domain that can each bind a TRBC polypeptide and administering the one or more molecules including a first antigen binding domain that can bind a TRBC polypeptide that is conjugated to one or more anti-cancer agents. In some cases, one or more bispecific molecules including a first antigen binding domain and a second antigen binding domain that can each bind a TRBC polypeptide can be administered to a mammal having a T-cell cancer, and one or more molecules including a first antigen binding domain that can bind a TRBC polypeptide that is conjugated to one or more anti-cancer agents can be administered to the same mammal if/when any relapse of the T-cell cancer is observed in the mammal. For example, one or more bispecific molecules including a first antigen binding domain and a second antigen binding domain that can each bind a TRBC polypeptide can be administered to a mammal having a T-cell cancer, and one or more molecules including a first antigen binding domain that can bind a TRBC polypeptide that is conjugated to one or more anti-cancer agents can be administered to the same mammal from immediately after a relapse of the T-cell cancer is observed in the mammal to years after a relapse of the T-cell cancer is observed in the mammal. For example, administration of one or more bispecific molecules including a first antigen binding domain and a second antigen binding domain that can each bind a TRBC polypeptide to a mammal and an administration of one or more molecules including a first antigen binding domain that can bind a TRBC polypeptide that is conjugated to one or more anti-cancer agents to the same mammal can be administered with from about 30 days to about 10 years apart separating the administrations.

In some cases, one or more molecules including a first antigen binding domain that can bind a TRBC polypeptide that is conjugated to one or more anti-cancer agents (e.g., a TRBC-ADC) can be administered to a mammal (e.g., a human) having a T cells cancer independent of one or more bispecific molecules provided herein (e.g., one or more bispecific molecules including a first antigen binding domain and a second antigen binding domain that can each bind a TRBC polypeptide and, optionally, one or more bispecific molecules including a first antigen binding domain that can bind a TRBC polypeptide and a second antigen binding domain that can bind a T cell co-receptor polypeptide such as a CD3 polypeptide). For example, one or more molecules including a first antigen binding domain that can bind a TRBC polypeptide that is conjugated to one or more anti-cancer agents can be used as the sole active agent to treat a mammal (e.g., a human) having a T cell cancer.

In some cases, the methods and materials described herein can include one or more (e.g., one, two, three, four, five or more) additional treatments (e.g., therapeutic interventions) that are effective to treat T cell cancers. For example, a mammal in need thereof (e.g., a mammal having a T cell cancer) can be administered one or more bispecific molecules provided herein (e.g., one or more bispecific molecules including a first antigen binding domain and a second antigen binding domain that can each bind a TRBC polypeptide and, optionally, one or more bispecific molecules including a first antigen binding domain that can bind a TRBC polypeptide and a second antigen binding domain that can bind a T cell co-receptor polypeptide such as a CD3 polypeptide) in combination with one or more therapeutic interventions. Examples of therapeutic interventions that can be used as described herein to treat a T cell cancer include, without limitation, cancer surgeries, radiation therapies, blood transplants (e.g., autologous blood transplants and allogeneic blood transplants), bone marrow transplants (e.g., autologous bone marrow transplants and allogeneic bone marrow transplants), and any combinations thereof. In some cases, the one or more additional treatments that are effective to treat T cell cancers can be performed at the same time as the administration of the one or more bispecific molecules provided herein. In some cases, the one or more additional treatments that are effective to treat T cell cancers can be performed before and/or after the administration of the one or more bispecific molecules provided herein.

In some cases, one or more bispecific molecules provided herein (e.g., bispecific molecules including a first antigen binding domain and a second antigen binding domain that can each bind a TRBC polypeptide and, optionally, bispecific molecules including a first antigen binding domain that can bind a TRBC polypeptide and a second antigen binding domain that can bind a T cell co-receptor polypeptide such as a CD3 polypeptide) can be used to treat a mammal having a clonal T cell expansion (e.g., a pathogenic clonal T cell expansions) other than cancer. For example, a mammal having a disease, disorder, or condition other than a T cell cancer that is associated with a clonal T cell expansion can be administered one or more bispecific molecules provided herein. In some cases, a disease, disorder, or condition other than a T cell cancer that is associated with a clonal T cell expansion can be an autoimmune disease. In some cases, a disease, disorder, or condition other than a T cell cancer that is associated with a clonal T cell expansion can be associated with transplant rejection. Examples of diseases, disorders, and conditions associated with a clonal T cell expansion that can be targeted using one or more bispecific molecules provided herein include, without limitation, graft versus host disease (GVHD), celiac disease, Felty's syndrome, Sjogren's syndrome, scleroderma, eosinophilic fasciitis, scleromyxedema, myositis, multiple sclerosis, Rasmussen's encephalitis, autoimmune thyroid diseases, neuromyelitis optica, aplastic anemia, paroxysmal nocturnal hemoglobinuria, Alzheimer's disease, narcolepsy, and aging. For example, one or more bispecific molecules provided herein (e.g., bispecific molecules including a first antigen binding domain and a second antigen binding domain that can each bind a TRBC polypeptide and, optionally, bispecific molecules including a first antigen binding domain that can bind a TRBC polypeptide and a second antigen binding domain that can bind a T cell co-receptor polypeptide such as a CD3 polypeptide) can be can be used to selectively deplete the clonally expanded T cells while sparing a fraction of healthy T cells (e.g., a number of healthy T cells sufficient to maintain adequate T cell immunity).

The invention will be further described in the following examples, which do not limit the scope of the invention described in the claims.

### EXAMPLES

### Example 1: TCR Beta Chain Constant Region-Targeting Antibodies for the Treatment of T Cell Malignancies

Antibodies directed against the pan-B cell markers CD19 or CD20 have demonstrated success in treating B cell malignancies. Such therapies result in the loss of healthy B cells, but this depletion is usually well-tolerated by patients. While analogous targeting of pan-T cell markers may help control T cell malignancies, the accompanying healthy T cell depletion would result in severe and unacceptable immunosuppression.

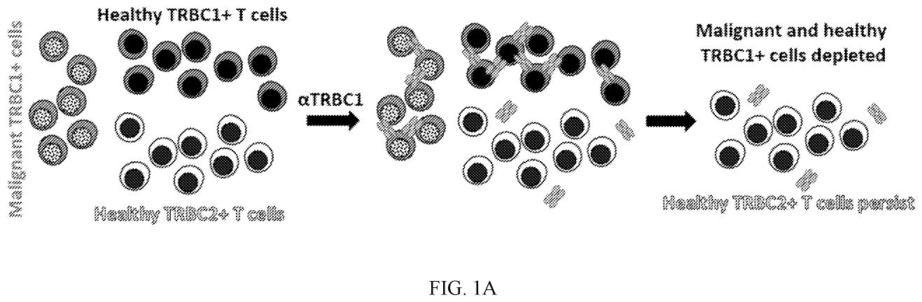

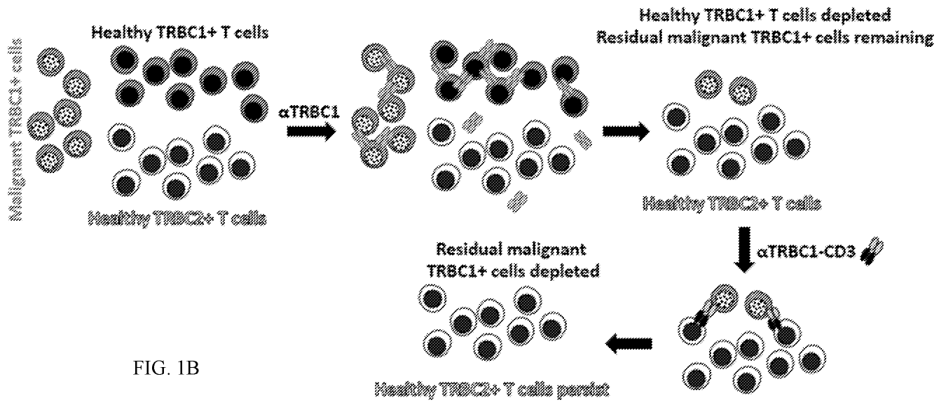

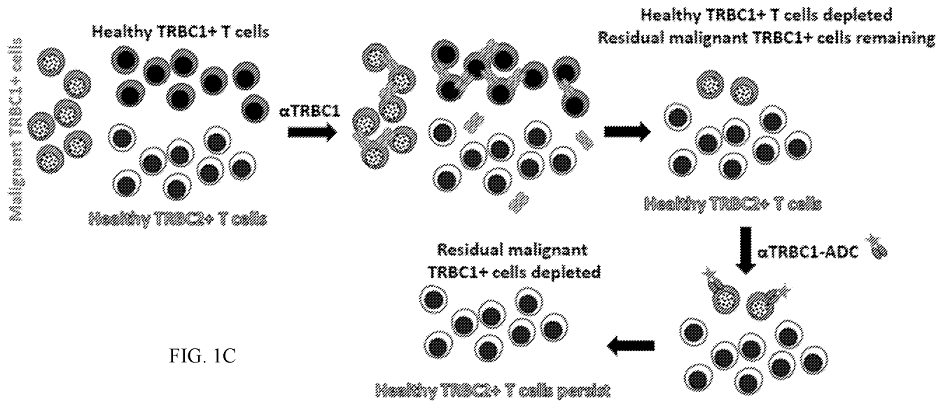

This Example describes the generation and evaluation of a TRBC targeting BsAbs for the treatment of T cell cancers. TRBC-targeting BsAbs can selectively target and deplete cancerous T cells. A bispecific antibody targeting TRBC1 can selectively deplete TRBC1⁺ T cell cancers and TRBC1⁺ healthy human T cells while sparing the TRBC2⁺ healthy human T cells (FIG. 1A). The remaining TRBC2⁺ healthy T cells are sufficient to maintain a functioning immune system.

### Generation of Bispecific Antibodies for Selective Targeting of TRBC1⁺ Malignant T Cells

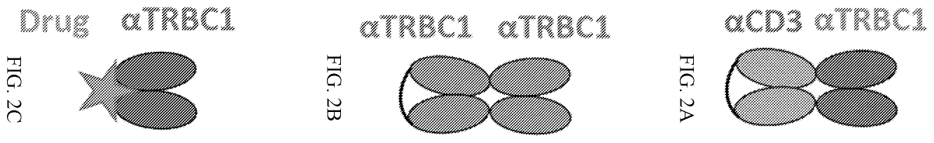

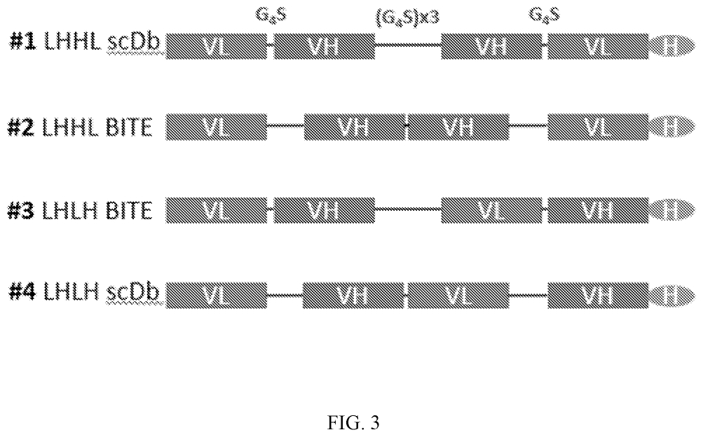

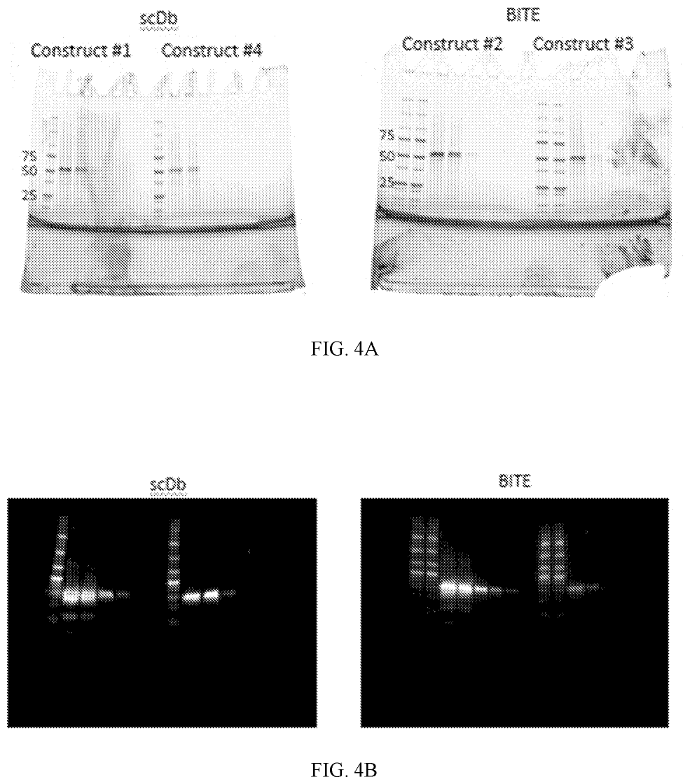

TRBC1-CD3 bispecific antibodies were generated by linking an anti-TRBC1 scFv to an anti-CD3 scFv (FIG. 2A). The TRBC1-CD3 bispecific antibody connects TRBC1⁺ T cell subset with all other T cells that express CD3. A second set of TRBC1-TRBC1 bispecific antibodies were generated by linking two anti-TRBC1 scFvs (FIG. 2B). The TRBC1-TRBC1 bispecific antibodies only connect a TRBC1⁺ T cell with another TRBC1⁺ T cell. The TRBC1-TRBC1 bispecific antibodies were generated using four distinct formats (FIG. 3). All bispecific antibodies were produced by HEK293F cell transfection, followed by purification using nickel affinity chromatography. Bispecific antibody expression was detected via SDS-PAGE gel electrophoresis (FIGS. 4A, 4B).

ScFv sequences

| | | | SEQ ID | | SEQ ID |
|---|---|---|---|---|---|
| scFv | Affinity | V_{L} | NO | V_{H} | NO |
| C1 | 0.4 nM | DVVMTQSPLSLPVSLGDQASISCRSSQR | 7 | EVRLQQSGPDLIKPGASVKMSCKASGYTFT | 8 |
| [symbol] ovi-1) | | LVHSNGNTYLHWYLQKPGQSPKLLIYRV | | GYVMHWVKQRPGQGLEWIGFINPYNDDIQS | |
| | | SNRFPGVPDRFSGSGSGTDFTLKISRVE | | NERFRGKATLTSDKSSTTAYMELSSLTSED | |
| | | AEDLGIYFCSQSTHVPYTFGGGTKLEIK | | SAVYYCARGAGYNFDGAYRFFDFWGQGTTL | |
| | | R | | TVSS | |
| UCHT1.v9) | 4.7 nM | DIQMTQSPSSLSASVGDRVTITCRASQD | 15 | EVQLVESGGGLVQPGGSLRLSCAASGYSFT | 16 |
| | | IRNYLNWYQQKPGKAPKLLIYYTSRLES | | GYTMNWVRQAPGKGLEWVALINPYKGVSTY | |
| | | GVPSRFSGSGSGTDYTLTISSLQPEDFA | | NQKFKDRFTISVDKSKNTAYLQMNSLRAED | |
| | | TYYCQQGNTLPWTFGQGTKVEIK | | TAVYYCARSGYYGDSDWYFDVWGQGTLVTV | |
| | | | | SS | |

[symbol]

indicates data missing or illegible when filed

### TRBC1-TRBC1 Bispecific Antibodies Bind to TRBC1⁺ Jurkat T Cells and not to the TRBC2⁺ HPB-ALL Cells

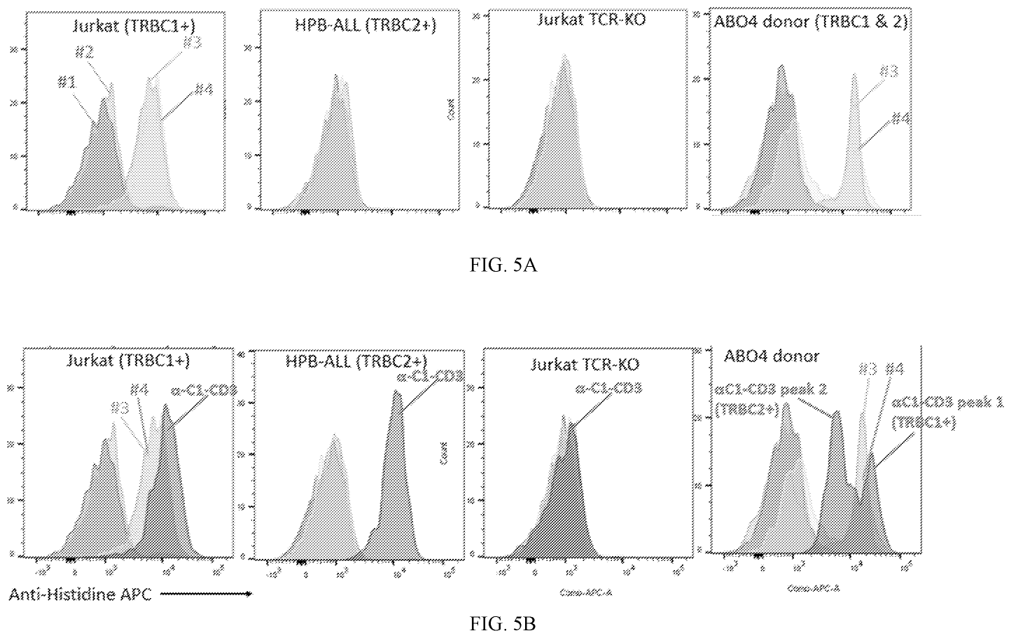

Human T cell cancer-derived cell lines have rearranged TCRβ genes and express either TRBC1 or TRBC2. The Jurkat cell line, derived from a T cell acute lymphoblastic leukemia (T-ALL) patient, expresses TRBC1. The HPB-ALL cell line, also derived from a different T-ALL patient, expresses TRBC2. Incubation of the four TRBC1-TRBC1 bispecific antibodies with Jurkat and HPB-ALL cell lines showed TRBC1-TRBC1 #3 and TRBC1-TRBC1 #4 bound Jurkat cells but not HPB-ALL cells (FIG. 5A). Jurkat cells with TCR gene knock out (TCR-KO) was used as negative control and did not show binding to any of the four TRBC1-TRBC1 bispecific antibodies. Thus TRBC1-TRBC1 #3 and TRBC1-TRBC1 #4 bispecific antibodies retained TRBC1 binding ability, while the TRBC1-TRBC1 #1 and TRBC1-TRBC1 #2 bispecific antibodies did not. Healthy polyclonal T cells obtained from a human donor (AB04) consists of both TRBC1 and TRBC2 subsets. TRBC1-TRBC1 #3 and TRBC1-TRBC1 #4 were able to bind a subset of healthy human T cells (FIG. 5A). TRBC1-CD3 bispecific antibody binding to Jurkat, HPB-ALL, and healthy human T cells obtained from the AB04 donor was also tested. TRBC1-CD3 bispecific antibody was able to bind both Jurkat cells (using the anti-TRBC1 scFv and anti-CD3 scFv) and HPB-ALL cells (using anti-CD3 scFv) (FIG. 5B). TRBC1-CD3 did not bind Jurkat TCR-KO cells as the cells lack both TRBC1 and CD3 on the cell surface. TRBC1-CD3 also showed binding to the healthy human T cells obtained from the AB04 donor at two different intensities; peak 1 and peak 2. This is likely because TRBC1-CD3 can bind TRBC1+ healthy human T cells using both the TRBC1 and the CD3 antigens, causing the higher intensity peak 1 stain. TRBC1-CD3 also bound TRBC2+ healthy human T cells using the CD3 antigen only, thus causing the lower intensity peak 2.

### TRBC1-TRBC1 Bispecific Antibodies Activate Healthy Human T Cells Against TRBC1⁺ T Cell Cancer Cell Line

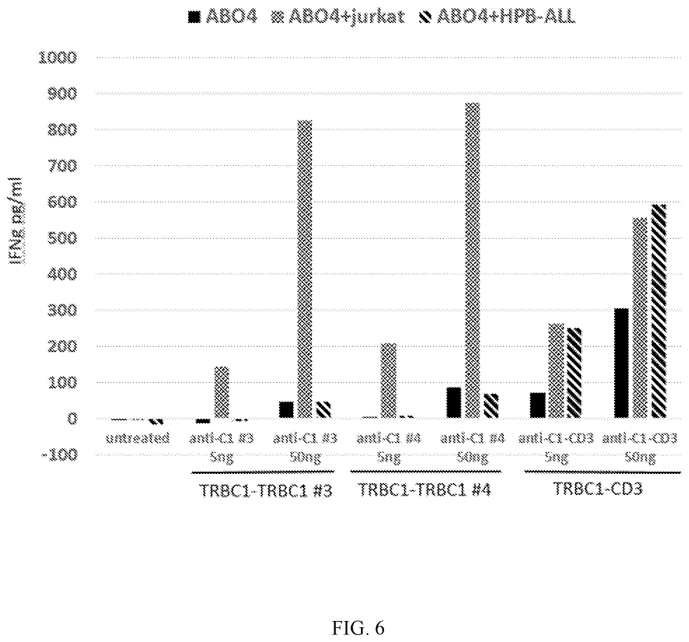

As the TRBC1-TRBC1 #3 and the TRBC1-TRBC1 #4 bispecific antibodies showed binding to Jurkat cells, the ability of these bispecific antibodies to induce healthy human T cell activation against T cell cancer cell lines was studied. To assess the activity of the TRBC1-TRBC1 bispecific antibodies and the TRBC1-CD3 bispecific antibody against T cell malignancies, healthy human T cells were co-cultured with T cell cancer cell lines in the presence or absence of different antibodies. Bispecific antibodies TRBC1-TRBC1 #3 and TRBC1-TRBC1 #4 demonstrated increased IFNγ production in presence of Jurkat cells but not HPB-ALL cells (FIG. 6). In contrast, TRBC1-CD3 bispecific antibody induced IFNγ secretion in presence of both Jurkat cells and HPB-ALL cells.

### TRBC1-TRBC1 Bispecific Antibodies Induce Healthy Human T Cells to Selectively Kill TRBC1⁺ T Cells and Spare the TRBC2⁺ T Cells

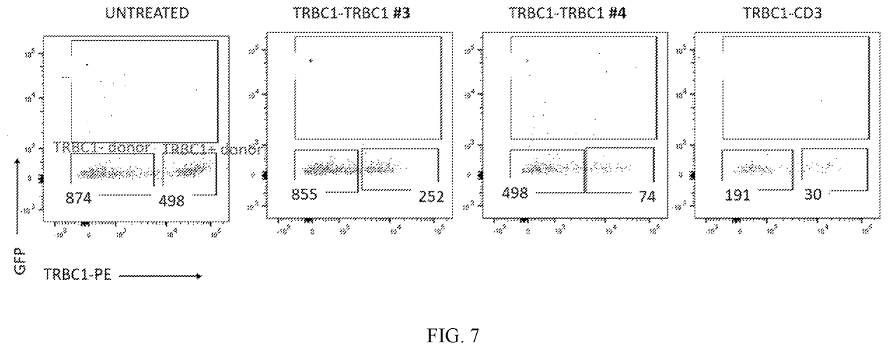

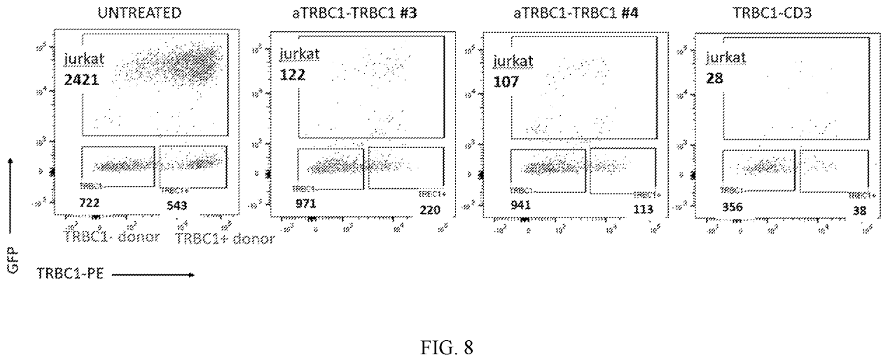

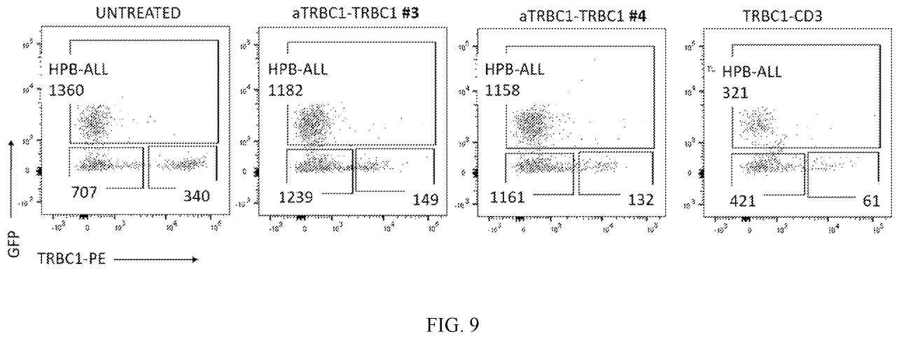

To assess cytotoxicity of the bispecific antibodies against healthy human T cells, healthy human T cells (AB04) were cultured in presence of the bispecific antibodies TRBC1-TRBC1 #3, TRBC1-TRBC1 #4, or the TRBC1-CD3 bispecific antibody. Exposure to TRBC1-TRBC1 #3 and TRBC1-TRBC1 #4 resulted in selective loss of healthy human TRBC1+ cell subset, without affecting the TRBC2⁺ T cell subset (FIG. 7). Conversely, TRBC1-CD3 bispecific antibody exposure lead to depletion of both TRBC1⁺ and TRBC2⁺ healthy human T cell subsets (FIG. 7). To test cytotoxicity of the bispecific antibodies against TRBC1⁺ T cell cancer cell line, healthy human T cells were co-cultured with Jurkat cells expressing GFP. In presence of the TRBC1-TRBC1 #3 or TRBC1-TRBC1 #4 bispecific antibody, depletion of Jurkat cells and TRBC1⁺ healthy T cells was observed, while the TRBC2⁺ healthy human T cells were preserved (FIG. 8). Similar co-culture in presence of TRBC1-CD3 showed depletion of Jurkat cells along with both TRBC1⁺ and TRBC2⁺ healthy human T cells (FIG. 8). This demonstrated that the TRBC1-TRBC1 #3 and TRBC1-TRBC1 #4 bispecific antibodies are capable of killing the TRBC1+ Jurkat cells, while sparing TRBC2⁺ healthy human T cells. Conversely, the TRBC1-CD3 bispecific antibody treatment lead to eradication of TRBC1⁺ Jurkat cells along with loss of both TRBC1⁺ and TRBC2⁺ healthy T cells, when there are TRBC1⁺ healthy T cells present. To test cytotoxicity of the bispecific antibodies against TRBC2⁺ T cell cancer cell line, healthy human T cells were co-cultured with GFP expressing HPB-ALL cells. Exposure to the TRBC1-TRBC1 #3 or the TRBC1-TRBC1 #4 bispecific antibodies failed to eradicate the HPB-ALL cells. A similar co-culture in the presence of TRBC1-CD3 bispecific antibody demonstrated depletion of HPB-ALL cells (FIG. 9).

### Elimination of Residual TRBC1⁺ Cancer Cells

TRBC1-TRBC1 bispecific antibodies can lead to killing of healthy TRBC1⁺ T cells by fratricide. It is possible that the TRBC1⁺ healthy T cells can be reduced to a number insufficient to finish their task before the TRBC1⁺ cancer cells are completely eliminated (FIG. 1B and FIG. 8). In such a scenario, a “mop up” therapy with the TRBC1-CD3 bispecific antibody can be used (FIG. 1B). As no or very few TRBC1⁺ healthy T cells are present, T cell activation mediated by the anti-TRBC1 portion of the TRBC1-CD3 bispecific antibody can be nonexistent or minimal, thus leaving the TRBC2⁺ T cell population mostly unharmed, which can carry out the remaining task of completely eliminating the residual TRBC1⁺ cancer cells.

A second “mop up” strategy may be provided (FIG. 1C) where therapy with a TRBC1 scFv or TRBC1 antibody conjugated to a toxin (a TRBC1-ADC) that eliminates the TRBC1⁺ malignant cells without the need for an effector T cell population. This strategy also leaves the healthy TRBC2⁺ T cell population unharmed, which can maintain cellular immunity.

### Summary

The TRBC1-TRBC1 #3 and the TRBC1-TRBC1 #4 bispecific antibodies bind only to TRBC1⁺ T cells, activate healthy TRBC1⁺ human T cells against TRBC1⁺ T cell cancers resulting in selective depletion of TRBC1⁺ cells while preserving the healthy TRBC2⁺ T cells. On the other hand, the TRBC1-CD3 bispecific antibody binds to both the TRBC1⁺ and TRBC2⁺ T cells, leading to near complete loss of all T cells. This demonstrates that TRBC1-TRBC1 #3 and the TRBC1-TRBC1 #4 bispecific antibodies are viable candidates for T cell cancer directed immunotherapy. In addition, a “mop up” strategy is proposed, in which the TRBC1-CD3 bispecific antibody is used to recruit the TRBC2⁺ healthy T cells for complete elimination of residual TRBC1⁺ cancer cells when the TRBC1⁺ healthy T cells are reduced to an insufficient number by the TRBC1-TRBC1 bispecific antibodies through fratricide. Alternatively, a TRBC1-ADC molecule can be used to kill the residual TRBC1⁺ malignant cells.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A bispecific molecule comprising:
a polypeptide comprising a first antigen binding domain that can bind a T cell receptor β chain constant region (TRBC) polypeptide; and
a polypeptide comprising a second antigen binding domain that can bind said TRBC polypeptide.

2. The bispecific molecule of claim 1, wherein said polypeptide comprising said first antigen binding domain that can bind said TRBC polypeptide and said polypeptide comprising said second antigen binding domain that can bind said TRBC polypeptide are each independently selected from the group consisting of a single-chain variable fragment (scFv), an antigen-binding fragment (Fab), a F(ab′)2 fragment, and biologically active fragments thereof.

3. The bispecific molecule of claim 1, wherein a binding affinity of said first antigen binding domain that can bind said TRBC polypeptide is lower than a binding affinity of said second antigen binding domain that can bind said TRBC polypeptide.

4. The bispecific molecule of claim 1, wherein said TRBC polypeptide is a TRBC1 polypeptide or a TRBC2 polypeptide.

5. The bispecific molecule of claim 4, wherein said TRBC polypeptide is said TRBC1 polypeptide.

6. The bispecific molecule of claim 5, wherein said first antigen binding domain that can bind to said TRBC1 polypeptide or said second antigen binding domain that can bind to said TRBC1 polypeptide comprises:
a light chain including a V_{L} CDR1 having an amino acid sequence set forth in SEQ ID NO:1, a V_{L} CDR2 having an amino acid sequence set forth in SEQ ID NO:2, and a V_{L} CDR3 having an amino acid sequence set forth in SEQ ID NO:3; and
a heavy chain including a V_{H} CDR1 having an amino acid sequence set forth in SEQ ID NO:4, a V_{H} CDR2 having an amino acid sequence set forth in SEQ ID NO:5, and a V_{H} CDR3 having an amino acid sequence set forth in SEQ ID NO:6.

7. The bispecific molecule of claim 6, wherein said light chain comprises an amino acid sequence set forth in SEQ ID NO:7, and wherein said heavy chain comprises an amino acid sequence set forth in SEQ ID NO:8.

8. The bispecific molecule of claim 6, wherein said light chain comprises an amino acid sequence set forth in SEQ ID NO:48, and wherein said heavy chain comprises an amino acid sequence set forth in SEQ ID NO:49.

9. The bispecific molecule of claim 1, wherein said bispecific molecule further comprises a molecule that can improve the stability of said bispecific molecule.

10. A method for treating a mammal having a T cell cancer, said method comprising administering to said mammal a bispecific molecule comprising:
a polypeptide comprising a first antigen binding domain that can bind a TRBC polypeptide; and
a polypeptide comprising a second antigen binding domain that can bind said TRBC polypeptide.

11. The method of claim 10, wherein said mammal is a human.

12. The method of claim 10, wherein said T cell cancer is a clonal T cell cancer.

13. The method of claim 10, wherein said T cell cancer is selected from the group consisting of acute lymphoblastic leukemia (ALL), peripheral T cell lymphomas (PTCL), angioimmunoblastic T cell lymphomas (AITL), T cell prolymphocytic leukemia (T-PLL), adult T cell leukemia/lymphoma (ATLL), enteropathy-associated T-cell lymphoma (EATL), monomorphic epitheliotropic intestinal T-cell lymphoma (MEITL), follicular T-cell lymphoma (FTCL), nodal peripheral T-cell lymphoma (nodal PTCL), cutaneous T cell lymphomas (CTCL), anaplastic large cell lymphoma (ALCL), T-cell large granular lymphocytic leukemia (T-LGL), extra nodal NK/T-Cell lymphoma (NKTL), and hepatosplenic T-cell lymphoma.

14. The method of claim 10, wherein said cancer cells within said mammal are reduced by at least 50 percent.

15. The method of claim 10, wherein said method is effective to improve survival of said mammal.

16. The method of claim 10, said method further comprising administering to said mammal, after said administration of said bispecific molecule, a second bispecific molecule comprising:
a polypeptide comprising a third antigen binding domain that can bind said TRBC polypeptide; and
a polypeptide comprising an antigen binding domain that can bind a CD3 polypeptide.

17. The method of claim 16, wherein said CD3 polypeptide is selected from the group consisting of a CD3γ polypeptide, a CD3δ polypeptide, and a CD3ε polypeptide.

18. The method of claim 10, said method further comprising administering to said mammal, after said administration of said bispecific molecule, a molecule comprising:
a polypeptide comprising a third antigen binding domain that can bind said TRBC polypeptide; and
an anti-cancer agent.

19-20. (canceled)

21. A method for treating a mammal having a T cell cancer, said method comprising:
administering to said mammal a first bispecific molecule comprising:
a polypeptide comprising a first antigen binding domain that can bind a TRBC polypeptide; and
a polypeptide comprising a second antigen binding domain that can bind said TRBC polypeptide; and
administering to said mammal a molecule comprising:
a polypeptide comprising a third antigen binding domain that can bind said TRBC polypeptide; and
an anti-cancer agent.

22. The method of claim 21, wherein said mammal is a human.

23. The method of claim 21, wherein said T cell cancer is a clonal T cell cancer.

24. The method of claim 21, wherein said T cell cancer is selected from the group consisting of ALL, PTCL, AITL, T-PLL, ATLL, EATL, MEITL, FTCL, nodal PTCL, CTCL, ALCL, T-LGL, extra nodal NKTL, and hepatosplenic T-cell lymphoma.

25. The method of claim 21, wherein said cancer cells within said mammal are reduced by at least 50 percent.

26. The method of claim 21, wherein said method is effective to improve survival of said mammal.

27. A method for treating a mammal having a disease, disorder, or condition associated with a clonal T cell expansion, said method comprising administering to said mammal a bispecific molecule comprising:
a polypeptide comprising a first antigen binding domain that can bind a TRBC polypeptide; and
a polypeptide comprising a second antigen binding domain that can bind said TRBC polypeptide.

28. The method of claim 27, wherein said mammal is a human.

29. The method of claim 27, wherein said disease, disorder, or condition associated with a clonal T cell expansion is selected from the group consisting of graft versus host disease (GVHD), celiac disease, Felty's syndrome, Sjogren's syndrome, scleroderma, eosinophilic fasciitis, scleromyxedema, myositis, multiple sclerosis, Rasmussen's encephalitis, autoimmune thyroid diseases, neuromyelitis optica, aplastic anemia, paroxysmal nocturnal hemoglobinuria, Alzheimer's disease, narcolepsy, and aging.

30. The method of claim 27, said method further comprising administering to said mammal, after said administration of said bispecific molecule, a second bispecific molecule comprising:
a polypeptide comprising a third antigen binding domain that can bind said TRBC polypeptide; and
a polypeptide comprising an antigen binding domain that can bind a CD3 polypeptide.

31. The method of claim 30, wherein said CD3 polypeptide is selected from the group consisting of a CD3γ polypeptide, a CD3δ polypeptide, and a CD3ε polypeptide.


SEQUENCE LISTING (49 sequences)

SEQ ID NO: 1 - protein, 16 aa, artificial
  anti-TRBC1 VL CDR1
RSSQRLVHSNGNTYLH

SEQ ID NO: 2 - protein, 7 aa, artificial
  anti-TRBC1 VL CDR2
RVSNRFP

SEQ ID NO: 3 - protein, 9 aa, artificial
  anti-TRBC1 VL CDR3
SQSTHVPYT

SEQ ID NO: 4 - protein, 7 aa, artificial
  anti-TRBC1 VH CDR1
GYTFTGY

SEQ ID NO: 5 - protein, 6 aa, artificial
  anti-TRBC1 VH CDR2
NPYNDD

SEQ ID NO: 6 - protein, 15 aa, artificial
  anti-TRBC1 VH CDR3
GAGYNFDGAYRFFDF

SEQ ID NO: 7 - protein, 113 aa, artificial
  anti-TRBC1 VL
DVVMTQSPLSLPVSLGDQASISCRSSQRLVHSNGNTYLHWYLQKPGQSPKLLIYRVSNRFPGVPDRFSGSGSGTDFTLKISRVEAEDLGIYFCSQSTHVPYTFGGGTKLEIKR

SEQ ID NO: 8 - protein, 124 aa, artificial
  anti-TRBC1 VH
EVRLQQSGPDLIKPGASVKMSCKASGYTFTGYVMHWVKQRPGQGLEWIGFINPYNDDIQSNERFRGKATLTSDKSSTTAYMELSSLTSEDSAVYYCARGAGYNFDGAYRFFDFWGQGTTLTVSS

SEQ ID NO: 9 - protein, 11 aa, artificial
  anti-CD3 VL CDR1
RASQDIRNYLN

SEQ ID NO: 10 - protein, 8 aa, artificial
  anti-CD3 VL CDR2
  VARIANT (1)..(1): Y at residue 1 is optional
YYTSRLHS

SEQ ID NO: 11 - protein, 9 aa, artificial
  anti-CD3 VL CDR3
QQGNTLPWT

SEQ ID NO: 12 - protein, 5 aa, artificial
  anti-CD3 VH CDR1
GYTMN

SEQ ID NO: 13 - protein, 17 aa, artificial
  anti-CD3 VH CDR2
LINPYKGVSTYNQKFKD

SEQ ID NO: 14 - protein, 13 aa, artificial
  anti-CD3 VH CDR3
SGYYGDSDWYFDV

SEQ ID NO: 15 - protein, 107 aa, artificial [sequence omitted]

SEQ ID NO: 16 - protein, 122 aa, artificial [sequence omitted]

SEQ ID NO: 17 - protein, 107 aa, artificial
  anti-CD3 UCHT1 VL
DIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIK

SEQ ID NO: 18 - protein, 122 aa, artificial
  anti-CD3 UCHT1 VH
EVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGAGTTVTVSS

SEQ ID NO: 19 - protein, 10 aa, artificial
  anti-CD3 VL CDR1
RASSSVSYMN

SEQ ID NO: 20 - protein, 10 aa, artificial
  anti-CD3 VL CDR1
SASSSVSYMN

SEQ ID NO: 21 - protein, 14 aa, artificial
  anti-CD3 VL CDR1
RSSTGAVTTSNYAN

SEQ ID NO: 22 - protein, 10 aa, artificial
  anti-CD3 VL CDR1
RASQSVSYMN

SEQ ID NO: 23 - protein, 7 aa, artificial
  anti-CD3 VL CDR2
DTSKVAS

SEQ ID NO: 24 - protein, 7 aa, artificial
  anti-CD3 VL CDR2
DTSKLAS

SEQ ID NO: 25 - protein, 7 aa, artificial
  anti-CD3 VL CDR2
GTNKRAP

SEQ ID NO: 26 - protein, 9 aa, artificial
  anti-CD3 VL CDR3
QQWSSNPLT

SEQ ID NO: 27 - protein, 9 aa, artificial
  anti-CD3 VL CDR3
QQWSSNPFT

SEQ ID NO: 28 - protein, 9 aa, artificial
  anti-CD3 VL CDR3
ALWYSNLWV

SEQ ID NO: 29 - protein, 5 aa, artificial
  anti-CD3 VH CDR1
RYTMH

SEQ ID NO: 30 - protein, 5 aa, artificial
  anti-CD3 VH CDR1
TYAMN

SEQ ID NO: 31 - protein, 16 aa, artificial
  anti-CD3 VH CDR2
YINPSRGYTNYNQKFK

SEQ ID NO: 32 - protein, 19 aa, artificial
  anti-CD3 VH CDR2
RIRSKYNNYATYYADSVKD

SEQ ID NO: 33 - protein, 17 aa, artificial
  anti-CD3 VH CDR2
YINPSRGYTNYADSVKG

SEQ ID NO: 34 - protein, 10 aa, artificial
  anti-CD3 VH CDR3
YYDDHYCLDY

SEQ ID NO: 35 - protein, 14 aa, artificial
  anti-CD3 VH CDR3
HGNFGNSYVSWFAY

SEQ ID NO: 36 - protein, 106 aa, artificial
  anti-CD3 L2K-07 VL
DIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 37 - protein, 119 aa, artificial
  anti-CD3 L2K-07 VH
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSS

SEQ ID NO: 38 - protein, 106 aa, artificial [sequence omitted]

SEQ ID NO: 39 - protein, 119 aa, artificial [sequence omitted]

SEQ ID NO: 40 - protein, 109 aa, artificial
  anti-CD3 hXR32 VL
QAVVTQEPSLTVSPGGTVTLTCRSSTGAVTTSNYANWVQQKPGQAPRGLIGGTNKRAPWTPARFSGSLLGGKAALTITGAQAEDEADYYCALWYSNLWVFGGGTKLTVL

SEQ ID NO: 41 - protein, 125 aa, artificial
  anti-CD3 hXR32 VH
EVQLVESGGGLVQPGGSLRLSCAASGFTFNTYAMNWVRQAPGKGLEWVARIRSKYNNYATYYADSVKDRFTISRDDSKNSLYLQMNSLKTEDTAVYYCVRHGNFGNSYVSWFAYWGQGTLVTVSS

SEQ ID NO: 42 - protein, 106 aa, artificial
  anti-CD3 diL2K VL
DIVLTQSPATLSLSPGERATLSCRASQSVSYMNWYQQKPGKAPKRWIYDTSKVASGVPARFSGSGSGTDYSLTINSLEAEDAATYYCQQWSSNPLTFGGGTKVEIK

SEQ ID NO: 43 - protein, 119 aa, artificial
  anti-CD3 diL2K VH
DVQLVQSGAEVKKPGASVKVSCKASGYTFTRYTMHWVRQAPGQGLEWIGYINPSRGYTNYADSVKGRFTITTDKSTSTAYMELSSLRSEDTATYYCARYYDDHYCLDYWGQGTTVTVSS

SEQ ID NO: 44 - protein, 5 aa, artificial
  polypeptide linker
GGGGS

SEQ ID NO: 45 - protein, 15 aa, artificial
  polypeptide linker
GGGGSGGGGSGGGGS

SEQ ID NO: 46 - protein, 18 aa, artificial
  polypeptide linker
VEGGSGGSGGSGGSGGVD

SEQ ID NO: 47 - protein, 6 aa, artificial
  polyhistidine tag
HHHHHH

SEQ ID NO: 48 - protein, 112 aa, artificial
  anti-TRBC1 VL
DIVMTQSPLSLPVTPGEPASISCRSSQRLVHSNGNTYLHWYLQKPGQSPRLLIYRVSNRFPGVPDRFSGSGSGTDFTLKISRVEAEDVGVYYCSQSTHVPYTFGQGTKLEIK

SEQ ID NO: 49 - protein, 124 aa, artificial
  anti-TRBC1 VH
QVQLVQSGAEVKKPGASVKVSCKASGYTFTGYVMHWVRQAPGQGLEWMGFINPYNDDIQSNERFRGRVTMTRDTSISTAYMELSRLRSDDTAVYYCARGAGYNFDGAYRFFDFWGQGTMVTVSS